(19) United States
(12) Patent Application Publication
(10) Pub. No.: US 2021/0020299 A1
(43) Pub. Date: Jan. 21, 2021

(54) DIET PLAN MANAGEMENT DEVICE, DIET PLAN MANAGEMENT SYSTEM, DIET PLAN MANAGEMENT METHOD, AND PROGRAM

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 17/031,427

(22) Filed: Sep. 24, 2020

Related U.S. Application Data
(63) Continuation of application No. PCT/JP2019/012125, filed on Mar. 22, 2019.

(30) Foreign Application Priority Data
Mar. 27, 2018 (JP) 2018-060601

(51) Int. Cl. G16H 20/60; G16H 10/60; G16H 50/30; G16H 50/70; G16H 50/20; G16H 40/67; G16H 40/20; G06Q 10/08; G16H 10/40; A61B 5/00; G09B 19/00

(52) U.S. Cl. CPC G16H 20/60; G16H 10/60; G16H 50/30; G16H 50/70; G16H 50/20; G09B 19/0092; G16H 40/20; G06Q 10/083; G16H 10/40; A61B 5/4872; G16H 40/67

(57) ABSTRACT

Provided are a diet plan management device, a diet plan management system, a diet plan management method, and a program capable of managing a diet plan in which the blood inspection is combined. A physical information acquisition unit that acquires physical information of a target person, a blood inspection item value acquisition unit that acquires a blood inspection item value of the target person, a diet plan creation unit that changes, based on a relationship between a first blood inspection value corresponding to first physical information which is the physical information before the target person implements a diet and a second blood inspection item value corresponding to second physical information as a target, the physical information to the second physical information from the first physical information and creates a diet plan for implementing one or more blood inspections, and a diet plan monitoring unit that monitors the diet plan based on a third blood inspection item value acquired from the blood inspection are provided.

### CROSS-REFERENCE TO RELATED APPLICATIONS

The present application is a Continuation of PCT International Application No. PCT/JP2019/012125 filed on Mar. 22, 2019 claiming priority under 35 U.S.C. § 119(a) to Japanese Patent Application No. 2018-060601 filed on Mar. 27, 2018. Each of the above applications is hereby expressly incorporated by reference, in its entirety, into the present application.

### BACKGROUND OF THE INVENTION

### 1. Field of the Invention

The present invention relates to a diet plan management device, a diet plan management system, a diet plan management method, and a program, and particularly to management of a diet plan.

### 2. Description of the Related Art

A world obesity population has reached 2.1 billion in 2013. The world obesity population has increased 2.5 times compared to 1980. It has known that obesity increases the risk of lifestyle-related diseases such as cerebrovascular disease, heart disease, and type 2 diabetes.

A diet program that uses input of information such as daily weight and management using a management system via the Internet has been put into practical use.

A custom-made genetic diet program that classifies weight gain of an individual by specifying genetic information unique to the individual and provides a supplement suitable for the classification of the weight gain of the individual based on a genetic inspection using saliva as a sample has been known. An abbreviation SNPs of English notation single nucleotide polymorphisms may be used as a term indicating the genetic information unique to the individual.

A system using a genetic inspection kit in a case where genetic information unique to the individual is specified has been known. An inspection applicant purchases the genetic inspection kit, collects a sample by using the genetic inspection kit, and delivers the collected sample to a genetic inspection institution. The inspection applicant can receive a genetic inspection result from the genetic inspection institution.

JP2007-034845A describes a health support information providing device that provides health support information suitable for a constitution of a user based on a plurality of genetic polymorphism information of the user. The health support information providing device described in JP2007-034845A specifies a genetic polymorphism of an obesity gene of the user by using a DNA inspection, and decides health support information corresponding to the specified genetic polymorphism information. The health support information includes any of information regarding meals, information regarding exercises, and information regarding dietary supplements such as supplements. DNA is an abbreviation for deoxyribonucleic acid.

JP2006-215662A describes a health encouragement program information providing device. The health encouragement program information providing device described in JP2006-215662A prescribes a blood pressure value as a condition for providing the health encouragement program, and provides the health encouragement program for hypertension according to the provided condition.

### SUMMARY OF THE INVENTION

A weight, a BMI value, and a body fat percentage are used as an index for grasping an effect of a diet. The weight, BMI value, and body fat percentage can be routinely measured, and the effect of the diet can be grasped based on the measurement result. BMI is an abbreviation for body mass index.

The diet is implemented for reducing the risk of the lifestyle-related diseases. For example, the risk of the cerebrovascular disease can be grasped by using a cholesterol value in a blood inspection. The risk of the type 2 diabetes can be grasped by using a blood sugar value and an HbA1c value in the blood inspection. HbA1c indicates glycohemoglobin.

However, even though the weight is routinely measured in order to grasp the effect of the diet, it is difficult to directly grasp whether or not the risk of the lifestyle-related diseases is lowered from the measurement result of the weight.

Although the invention described in JP2007-034845A can provide the health support information suitable for the constitution of the user based on the genetic polymorphism information, the invention described in JP2007-034845A is difficult to grasp the progress of the diet and the effect of the diet in a case where the user takes measures such as meals and exercises.

Although the invention described in JP2006-215662A can guide the provision of the health encouragement program for hypertension and the suspension of the provision thereof according to the blood pressure value, it is difficult to grasp the progress of the diet and the effect of the diet.

The present invention has been made in view of the above circumstances, and an object thereof is to provide a diet plan management device, a diet plan management system, a diet plan management method, and a program capable of managing a diet plan in which the blood inspection is combined.

To achieve the above object, the following aspects of the invention are provided.

A diet plan management device according to a first aspect is a diet plan management device comprising a physical information acquisition unit that acquires physical information of a target person including at least one of a weight, a BMI value, or a body fat percentage of the target person, a blood inspection item value acquisition unit that acquires a blood inspection item value of the target person, a diet plan creation unit that changes, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creates a diet plan for implementing one or more blood inspections, and a diet plan monitoring unit that monitors the diet plan based on a third blood inspection item value which is acquired by using the blood inspection item value acquisition unit and is acquired as a result of the blood inspection.

According to the first aspect, the diet plan including one or more blood inspections is created. The diet plan is managed based on the third blood inspection item value obtained from the one or more blood inspections. Accordingly, it is possible to manage the diet plan in which the blood inspection is combined.

The first physical information may be acquired from a physical information storage unit that stores the physical information for each target person. The physical information storage unit may be connected to the diet plan management device so as to perform data communication.

The first blood inspection item value and the second blood inspection item value may be acquired from a blood inspection item value storage unit that stores the blood inspection item value associated with the physical information. The blood inspection item value storage unit may be connected to the diet plan management device so as to perform data communication.

The diet plan management device according to the first aspect is a diet plan management device comprising one or more processors and one or more memories. The processor acquires physical information which is physical information of a target person including at least one of a weight, a BMI value, or a body fat percentage of the target person, acquires a first blood inspection item value corresponding to first physical information before the target person implements a diet and a second blood inspection item value corresponding to second physical information as a target, changes the physical information of the target person to the second physical information from the first physical information and creates a diet plan for implementing one or more blood inspections based on a relationship between the first blood inspection item value and the second blood inspection item value, and monitors the diet plan based on a third blood inspection item value acquired as a result of the blood inspection. The memory can function as the diet plan management device that stores data in each processing.

According to a second aspect, in the diet plan management device of the first aspect, the diet plan creation unit may create the diet plan for implementing one or more measurements of the physical information, and the diet plan monitoring unit may monitor the diet plan based on third physical information acquired as a result of the measurement of the physical information.

According to the second aspect, the one or more measurements of the physical information is implemented. Accordingly, it is possible to manage the diet plan using the third physical information obtained from the measurement result of the physical information.

According to a third aspect, the diet plan management device of the second aspect may further comprise a physical information storage unit that stores the third physical information acquired from the measurement of the physical information.

According to the third aspect, it is possible to manage the diet plan using the third physical information read out from the physical information storage unit.

One or more storage elements can be applied to the physical information storage unit. A partial region of one storage element may be applied to the physical information storage unit.

According to a fourth aspect, in the diet plan management device of any one aspect of the first aspect to the third aspect, the physical information acquisition unit may acquire the second physical information, and the diet plan creation unit may create the diet plan by using the acquired second physical information.

According to the fourth aspect, it is possible to create the diet plan using the acquired second physical information.

The physical information storage unit may comprise a second physical information acquisition unit that acquires the second physical information.

According to a fifth aspect, in the diet plan management device of any one aspect of the first aspect to the third aspect, the diet plan creation unit may create the diet plan by using prescribed second physical information.

According to the fifth aspect, it is possible to create the diet plan using the prescribed second physical information.

As an example of the prescribed second physical information, there is the second physical information prescribed in the diet plan. The second physical information storage unit that stores the prescribed second physical information may be provided. The prescribed second physical information may be read out from the second physical information storage unit.

According to a sixth aspect, in the diet plan management device of the second aspect or the third aspect, the diet plan creation unit may decide at least one of an implementation period in which the diet plan is implemented, a measurement frequency of the third physical information, or an implementation frequency of the blood inspection.

According to the sixth aspect, it is possible to create the diet plan including at least one of the implementation period of the diet plan, the measurement frequency of the third physical information, or the implementation frequency of the blood inspection.

A plurality of diet plans that prescribes, as a set, the implementation period of the diet plan, the measurement frequency of the third physical information, and the implementation frequency of the blood inspection is prepared. A diet plan selection information acquisition unit that acquires selection information for selecting one diet plan from the plurality of diet plans may be provided.

According to a seventh aspect, the diet plan management device of the second aspect, the third aspect, or the sixth aspect may further comprise an analysis unit that creates progress status information indicating a progress status of the diet plan by analyzing at least one of the third physical information or the third blood inspection item value.

According to the seventh aspect, it is possible to create the progress status information indicating the progress status of the diet plan based on the analysis result of at least one of the third physical information or the third blood inspection item value.

According to an eighth aspect, the diet plan management device of the seventh aspect may further comprise a progress status information output unit that outputs the progress status information.

According to the eighth aspect, it is possible to grasp the progress status of the diet plan based on the progress status information.

The progress status information may be acquired by using a terminal device that is connected to communicate with the diet plan management device.

The progress status information may include an achievement level of the blood inspection item value. Both the achievement level of the blood inspection item value and the achievement level of the physical information may be included.

According to a ninth aspect, in the diet plan management device of the seventh aspect or the eighth aspect, the analysis unit may generate change proposal information indicating a change proposal of the diet plan based on the progress status information.

According to the ninth aspect, it is possible to create the change proposal of the diet plan based on the progress status information.

According to a tenth aspect, the diet plan management device of the ninth aspect may further comprise a change proposal information output unit that outputs the change proposal information.

According to the tenth aspect, the change proposal of the diet plan can be grasped based on the change proposal information.

The change proposal information may be acquired by using the terminal device that is connected to communicate with the diet plan management device. The terminal device may transmit selection information indicating whether or not to change the diet plan to the diet plan management device.

According to an eleventh aspect, the diet plan management device of any one aspect of the sixth aspect to the tenth aspect may further comprise a delivery information output unit that transmits, to a delivery source of a blood inspection kit, blood inspection kit delivery information for delivering the blood inspection kit used for the blood inspection to the target person based on the implementation frequency of the blood inspection.

According to the eleventh aspect, it is possible to deliver the blood inspection kit based on the implementation frequency of the blood inspection.

The blood inspection kit delivery information may be directly transmitted or may be indirectly transmitted from the diet plan management device to the delivery source of the blood inspection kit.

According to a twelfth aspect, in the diet plan management device of any one aspect of the sixth aspect to the eleventh aspect, in a case where an input of the third physical information based on an input frequency of the third physical information is not implemented, the diet plan monitoring unit may create encouragement information for encouraging the input of the third physical information.

According to the twelfth aspect, in a case where the input of the third physical information is delayed, it is possible to create the encouragement information for encouraging the input of the third physical information.

According to a thirteenth aspect, the diet plan management device of the twelfth aspect may further comprise an encouragement information output unit that outputs the encouragement information.

According to the thirteenth aspect, the delay in the input of the third physical information can be grasped based on the encouragement information.

The encouragement information may be acquired by using the terminal device that is connected to communicate with the diet plan management device.

According to a fourteenth aspect, in the diet plan management device of any one aspect of the first aspect to the thirteenth aspect, the diet plan creation unit may create the diet plan by using a result of an intestinal flora inspection together.

According to the fourteenth aspect, it is possible to create the diet plan using the result of the intestinal flora inspection together.

According to a fifteenth aspect, in the diet plan management device of any one aspect of the first aspect to the fourteenth aspect, the diet plan creation unit may create the diet plan by using a result of a genetic inspection together.

According to the fifteenth aspect, it is possible to create the diet plan using the result of the genetic inspection together.

According to a sixteenth aspect, in the diet plan management device of any one aspect of the first aspect to the fifteenth aspect, the blood inspection item value acquisition unit may acquire, as the first blood inspection item value, the second blood inspection item value, and the third blood inspection item value, at least one item value of blood sugar, HbA1c, HDL cholesterol, LDL cholesterol, neutral fat, AST, ALT, γ-GPT, ALB, ALP, or e-GFR.

According to the sixteenth aspect, it is possible to manage the diet plan using the third blood inspection item value such as blood sugar.

A diet plan management system according to a seventeenth aspect is a diet plan management system comprising a server device configured to be connected to a network. The server device includes a physical information acquisition unit that acquires physical information of a target person including at least one of a weight, a BMI value, or a body fat percentage of the target person, a blood inspection item value acquisition unit that acquires a blood inspection item value of the target person, a diet plan creation unit that changes, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creates a diet plan for implementing one or more blood inspections, and a diet plan monitoring unit that monitors the diet plan based on a third blood inspection item value which is acquired by using the blood inspection item value acquisition unit and is acquired as a result of the blood inspection.

According to the seventeenth aspect, the same effect as the effect in the first aspect can be obtained.

In the seventeenth aspect, matters similar to the matters specified in the second aspect to the sixteenth aspect can be appropriately combined. In this case, the components serving as the processing and functions specified in the diet plan management device can be grasped as the components of the diet plan management system serving as the corresponding processing and functions.

According to an eighteenth aspect, the diet plan management system of the seventeenth aspect may further comprise a physical information storage device that is connected to communicate with the server device via the network. The physical information storage device may store the physical information obtained from a measurement of the physical information.

According to the eighteenth aspect, it is possible to manage the diet plan using the third physical information read out from the physical information storage unit connected to communicate with the server device.

According to a nineteenth aspect, the diet plan management system of the seventeenth aspect or the eighteenth aspect may further comprise a blood inspection item value storage device that is connected to communicate with the server device via the network. The blood inspection item value storage device may store the blood inspection item value associated with the physical information.

According to the nineteenth aspect, it is possible to acquire the blood inspection item value from the blood inspection item value storage device connected to communicate with the server device via the network.

According to a twentieth aspect, in the diet plan management system of the nineteenth aspect, the blood inspection item value storage device may store a relationship between at least one of personal information, hobby and taste information regarding hobby and taste, living environment information regarding a living environment, or family information regarding a family and the blood inspection item value.

According to the twentieth aspect, it is possible to acquire the blood inspection item value reflecting the personal information.

According to a twenty-first aspect, the diet plan management system of any one aspect of the seventeenth aspect to the twentieth aspect may further comprise a terminal device that is connected to communicate with the server device via the network. The terminal device may include a display unit that displays information regarding the diet plan of the target person, and the display unit displays, as the information regarding the diet plan, at least one of an application screen for applying for the diet plan, a proposal screen representing a proposal content of the diet plan, or a progress screen representing a progress of the diet plan.

According to the twenty-first aspect, it is possible to grasp the information regarding the diet plan of the target person displayed on the display unit of the terminal device.

The display unit may display the change selection screen for selecting whether or not to change the diet plan. The display unit may display the physical information input screen for inputting the physical information.

A diet plan management method according to a twenty-second aspect is a diet plan management method comprising a physical information acquisition step of acquiring physical information of a target person including at least one of a weight, a BMI value, or a body fat percentage of the target person, a blood inspection item value acquisition step of acquiring a blood inspection item value of the target person, a diet plan creation step of changing, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creating a diet plan for implementing one or more blood inspections, and a diet plan monitoring step of monitoring the diet plan based on a third blood inspection item value which is acquired in the blood inspection item value acquisition step and is acquired as a result of the blood inspection.

According to the twenty-second aspect, the same effect as the effect in the first aspect can be obtained.

In the twenty-second aspect, matters similar to the matters specified in the second aspect to the sixteenth aspect and the eighteenth aspect to the twenty-first aspect can be appropriately combined. In this case, the components serving as the processing and functions specified in the diet plan management device and the diet plan management system can be grasped as the components of the diet plan management method serving as the corresponding processing and functions.

A program according to a twenty-third aspect is a program causing a computer to realize a physical information acquisition function of acquiring physical information of a target person including at least one of a weight, a BMI value, or a body fat percentage of the target person, a blood inspection item value acquisition function of acquiring a blood inspection item value of the target person, a diet plan creation function of changing, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creating a diet plan for implementing one or more blood inspections, and a diet plan monitoring function of monitoring the diet plan based on a third blood inspection item value which is acquired by using the blood inspection item value acquisition function and is acquired as a result of the blood inspection.

According to the twenty-third aspect, the same effect as the effect of the first aspect can be obtained.

In the twenty-third aspect, matters similar to the matters specified in the second aspect to the sixteenth aspect and the eighteenth aspect to the twenty-first aspect can be appropriately combined. In this case, the components serving as the processing and functions specified in the diet plan management device and the diet plan management system can be grasped as the components of the program serving as the corresponding processing and functions.

According to the present invention, the diet plan including one or more blood inspections is created. The diet plan is managed based on the third blood inspection item value indicating the results of one or more blood inspections. Accordingly, it is possible to manage the diet plan in which the blood inspection is combined.

### BRIEF DESCRIPTION OF THE DRAWINGS

FIG. 1 is a block diagram showing a configuration example of a diet plan management system according to a first embodiment.

FIG. 2 is a block diagram showing an example of a hardware configuration of a server device.

FIG. 3 is a functional block diagram of the server device.

FIG. 4 is a schematic diagram showing a configuration example of a personal information database.

FIG. 5 is a conceptual diagram of blood inspection item value acquisition using a big database.

FIG. 6 is a flowchart showing a procedure flow of a diet plan management method according to the first embodiment.

FIG. 7 is a flowchart showing a procedure flow of a diet plan creation step shown in FIG. 6.

FIG. 8 is a flowchart showing a procedure flow of a diet plan monitoring step shown in FIG. 6.

FIG. 9 is a flowchart showing a procedure flow of a third physical information acquisition step shown in FIG. 8.

FIG. 10 is a flowchart showing a procedure flow of a third blood inspection item value acquisition step shown in FIG. 8.

FIG. 11 is a flowchart showing a flow of information between components of the diet plan management system and a flow of a blood inspection kit.

FIG. 12 is an explanatory diagram showing an example of a diet application screen.

FIG. 13 is an explanatory diagram showing an example of a diet plan proposal screen.

FIG. 14 is an explanatory diagram showing an example of a diet acceptance screen.

FIG. 15 is an explanatory diagram showing an example of a weight information input screen.

FIG. 16 is an explanatory diagram showing an example of a progress report screen.

FIG. 17 is an explanatory diagram showing another example of the progress report screen.

FIG. 18 is a block diagram showing a configuration example of a diet plan management system according to a modification example of the first embodiment.

FIG. 19 is a flowchart showing a procedure flow of a third physical information acquisition step in a diet plan management method according to a second embodiment.

FIG. 20 is an explanatory diagram showing an example of a drop concern warning screen.

FIG. 21 is a flowchart showing a procedure flow of a diet plan management method according to a third embodiment.

FIG. 22 is an explanatory diagram showing an example of an option inspection application screen.

FIG. 23 is a block diagram showing a configuration example of a diet plan management system to which a network system is applied.

### DESCRIPTION OF THE PREFERRED EMBODIMENTS

Hereinafter, preferred embodiments of the present invention will be described in detail with reference to the accompanying drawings. In the present specification, the same components are denoted by the same references, and thus, the redundant description is appropriately omitted.

### First Embodiment

[Overall Configuration of Diet Plan Management System]

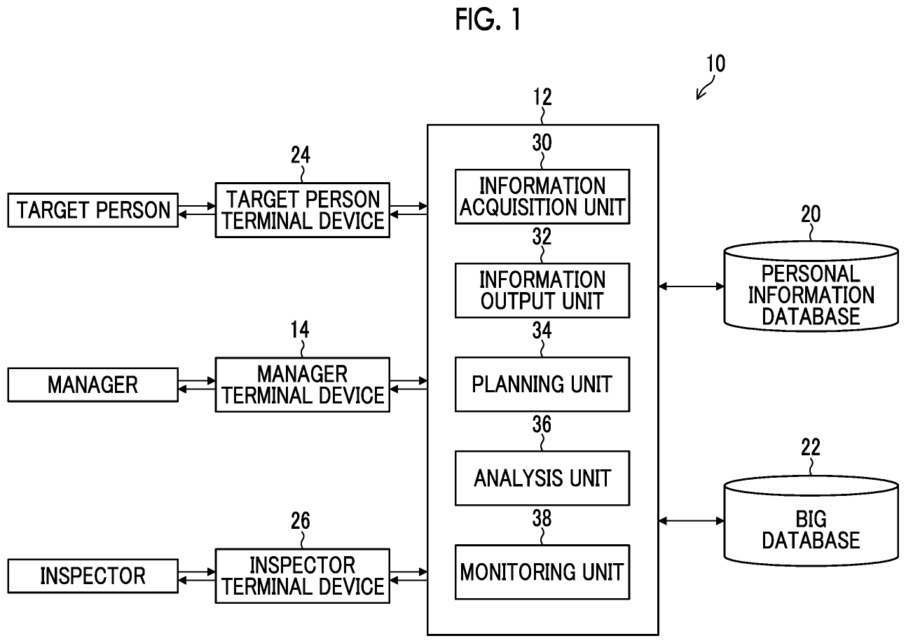

FIG. 1 is a block diagram showing a configuration example of a diet plan management system according to a first embodiment. A diet plan management system 10 shown in FIG. 1 comprises a server device 12 and a manager terminal device 14. The server device 12 and the manager terminal device 14 are connected so as to be able to perform data communication. The server device 12 corresponds to an example of a diet plan management device.

The server device 12 is connected to a personal information database 20, a big database 22, a target person terminal device 24, and an inspector terminal device 26 so as to be able to perform data communication. A well-known standard is applicable to the data communication in the present embodiment. The personal information database 20 corresponds to an example of a physical information storage unit and a physical information storage device. The big database 22 corresponds to an example of a blood inspection item value storage device.

The target person terminal device 24 is a terminal device operated by a target person. Examples of the target person terminal device 24 include a computer such as a personal computer and a communication terminal device such as a smartphone.

The inspector terminal device 26 is a terminal device arranged in an inspection institution (not shown), and is a terminal device operated by an inspector belonging to the inspection institution. Examples of the inspector terminal device 26 include a computer such as a personal computer.

The server device 12 comprises an information acquisition unit 30, an information output unit 32, a planning unit 34, an analysis unit 36, and a monitoring unit 38. The server device 12 comprises a storage unit (not shown). The information acquisition unit 30 acquires various kinds of information such as physical information of the target person transmitted by the target person via the target person terminal device 24. The information output unit 32 outputs various kinds of information such as diet result information.

In the present specification, for information and signals, output and transmission can be interchangeable, and acquisition and reception can be interchangeable.

The planning unit 34 creates a diet plan for each target person. Details of the creation of the diet plan for each target person using the planning unit 34 will be described later. The planning unit 34 corresponds to an example of a diet plan creation unit.

The analysis unit 36 analyzes the diet plan based on the physical information of the target person and a result of a blood inspection of the target person. Details of the analysis of the diet plan using the analysis unit 36 will be described later. The monitoring unit 38 monitors the diet plan implemented for each target person. Details of the monitoring of the diet plan using the monitoring unit 38 will be described later. The monitoring unit 38 corresponds to an example of a diet plan monitoring unit.

The manager terminal device 14 is a terminal device arranged in a management institution of the diet plan. The manager terminal device 14 is a terminal device operated by a manager belonging to the management institution of the diet plan. Examples of the manager terminal device 14 include a computer such as a personal computer.

[Hardware Configuration of Server Device]

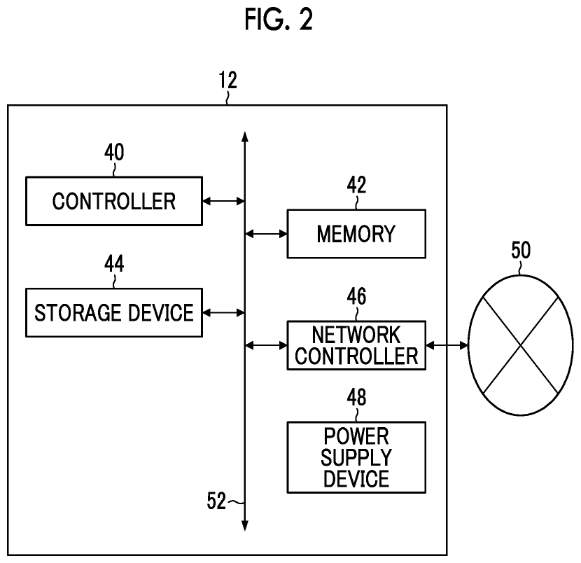

FIG. 2 is a block diagram showing an example of a hardware configuration of the server device. The server device 12 comprises a controller 40, a memory 42, a storage device 44, a network controller 46, and a power supply device 48. The controller 40, the memory 42, the storage device 44, and the network controller 46 are connected via a bus 52 so as to perform data communication.

<Controller>

The controller 40 functions as an overall controller, various calculation units, and a storage controller of the server device 12. The controller 40 executes a program stored in a read only memory (ROM) included in the memory 42.

The controller 40 downloads a program from an external storage device via the network controller 46, and executes the downloaded program. The external storage device may be connected so as to be able to communicate with the server device 12 via a network 50.

The controller 40 uses a random access memory (RAM) included in the memory 42 as a calculation region, and executes various kinds of processing in cooperation with various programs. Accordingly, various functions of the server device 12 are realized.

The controller 40 controls the reading-out of data from the storage device 44 and the writing of data to the storage device 44. The controller 40 may acquire various kinds of data from an external storage device via the network controller 46. The controller 40 can execute various kinds of processing such as calculation by using the various kinds of acquired data.

The controller 40 may include one or two or more processors. Examples of the processor include a field programmable gate array (FPGA) and a programmable logic device (PLD). Circuit configurations of the FPGA and the PLD are changeable after being manufactured.

Another example of the processor is an application specific integrated circuit (ASIC). The ASIC has a circuit configuration specifically designed in order to execute specific processing.

The controller 40 can apply two or more processors of the same kind. For example, the controller 40 may use two or more FPGAs, or may use two PLDs. The controller 40 may apply two or more processors of different kinds. For example, the controller 40 may apply one or more FPGAs and one or more ASICs.

In a case where a plurality of controllers is provided, the plurality of controllers may be constituted by using one processor. As an example of a case where the plurality of controllers is constituted by one processor, there is a form in which one processor is constituted by using a combination of one or more central processing units (CPUs) and software and the processor functions as the plurality of controllers. A graphics processing unit (GPU) which is a processor specialized in image processing may be applied instead of the CPU or in combination with the CPU. The software mentioned herein is synonymous with the program. As a representative example in which the plurality of controllers is constituted by using one processor, there is a computer such as a client device and a server device.

As another example in which the plurality of controllers is constituted by one processor, there is a form in which a processor that realizes the functions of the entire system including the plurality of controllers by one IC chip is used. A representative example of the processor that realizes the functions of the entire system including the plurality of controllers, there is a system on chip (SoC). IC is an abbreviation for an Integrated Circuit.

In this manner, the controller 40 is constituted by using as one or more of various processors as a hardware structure.

<Memory>

The memory 42 comprises a ROM (not shown) and a RAM (not shown). The ROM stores various programs executed by the server device 12. The ROM stores parameters used for executing various programs, and files. The RAM functions as a temporary storage region of data and a work region of the controller 40.

<Storage Device>

The storage device 44 stores various kinds of data in a non-transitory manner. The storage device 44 may be externally attached to the outside of the server device 12. A large-capacity semiconductor memory device may be applied instead of or in combination with the storage device 44.

<Network Controller>

The network controller 46 controls data communication with external devices such as the manager terminal device 14, the target person terminal device 24, and the inspector terminal device 26 shown in FIG. 1. The control of the data communication may include management of traffic for the data communication.

<Power Supply Device>

A large-capacity power supply device such as an uninterruptible power supply (UPS) is applied as the power supply device 48. In a case where a commercial power supply is cut off due to a power failure, the power supply device 48 supplies a power to the server device 12. The hardware configuration of the server device 12 shown in FIG. 2 is an example, and addition, deletion, and change can be appropriately made.

[Function of Server Device]

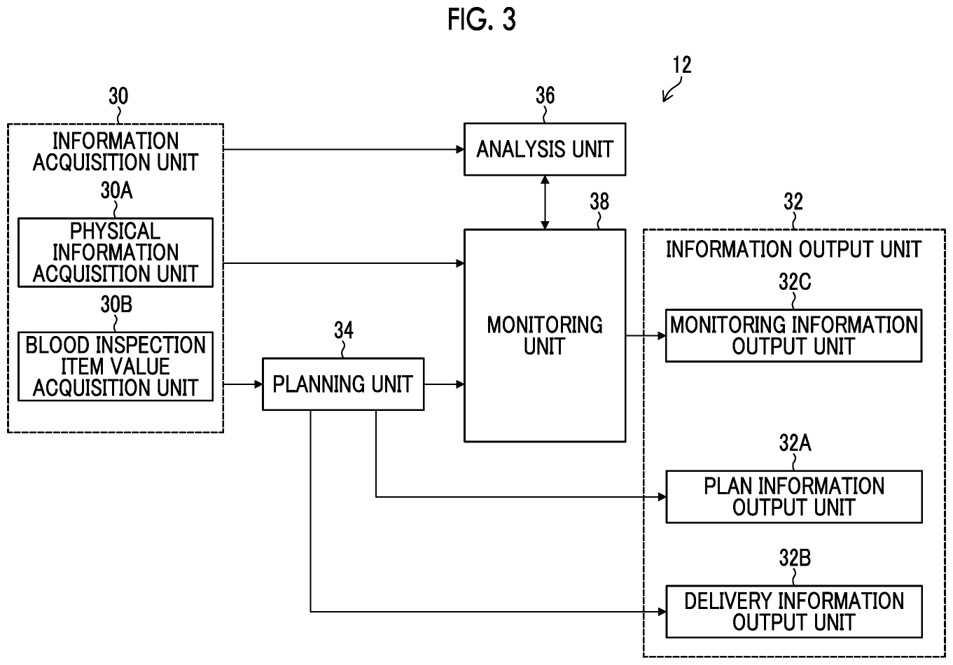

FIG. 3 is a functional block diagram of the server device. The server device 12 shown in FIG. 3 comprises the information acquisition unit 30, the information output unit 32, the planning unit 34, the analysis unit 36, and the monitoring unit 38. Hereinafter, the details of the units will be described.

<Information Acquisition Unit>

The information acquisition unit 30 comprises a physical information acquisition unit 30A and a blood inspection item value acquisition unit 30B. The information acquisition unit 30 may comprise various information acquisition units that acquire various kinds of information. The illustration of the various information acquisition units is omitted.

Individual hardware or common hardware may be applied to the physical information acquisition unit 30A and the blood inspection item value acquisition unit 30B. An example of a hardware configuration of the information acquisition unit 30, there is a configuration including a signal input element and a signal conversion element.

The physical information acquisition unit 30A acquires physical information including at least one of a weight of the target person, a BMI value, or a body fat percentage. The physical information acquisition unit 30A stores the physical information in a prescribed storage unit that is not shown in FIG. 2.

The physical information acquisition unit 30A acquires the physical information corresponding to the target person from the personal information database 20 shown in FIG. 1. In a case where the target person who desires to apply for a diet applies for diet, the physical information acquisition unit 30A can acquire first physical information output from the target person terminal device 24 shown in FIG. 1. The first physical information is physical information of the target person before the target person implements the diet.

The physical information acquisition unit 30A acquires second physical information. The second physical information is target physical information of the target person. Examples of the second physical information include a target weight, a target BMI value, and a target body fat percentage.

In a case where the first physical information is the weight, the physical information acquisition unit 30A acquires the target weight of the target person as the second physical information. In a case where the first physical information is the target BMI value, the physical information acquisition unit 30A acquires the target BMI value of the target person. In a case where the first physical information is the target body fat percentage, the physical information acquisition unit 30A acquires the target body fat percentage of the target person.

The prescribed second physical information may be applied as the second physical information. For example, in a case where the physical information is the weight and the diet plan is a minus 5-kilogram course, minus 5 kilograms is acquired as the second physical information from an initial weight that is the first physical information.

The physical information acquisition unit 30A acquires the third physical information. Third physical information is a measurement result of the physical information during the implementation of the diet plan, and is physical information stored in the personal information database 20.

The blood inspection item value acquisition unit 30B acquires blood inspection item values such as blood sugar, HbA1c, HDL cholesterol, LDL cholesterol, neutral fat, AST, ALT, γ-GPT, ALB, ALP, and e-GFR included in a blood inspection result. The blood inspection item value acquisition unit 30B stores the acquired blood inspection item value in a storage unit (not shown).

The blood inspection item value acquisition unit 30B acquires the blood inspection item value output from the inspector terminal device 26 shown in FIG. 1. The blood inspection item value acquisition unit 30B may acquire the blood inspection item value output from the target person terminal device 24 shown in FIG. 1.

HbA1c indicates glycohemoglobin. HDL is an abbreviation for high density lipoprotein. LDL is an abbreviation for low density lipoprotein.

AST indicates aspartate aminotransferase. ALT indicates alanine aminotransferase. γ-GPT indicates gamma glutamyl transpeptidase. ALB indicates albumin. ALP indicates alkaline phosphatase. e-GFR indicates an estimated glomerular filtration rate.

The information transmitted from the target person terminal device 24 in a case where the target person applies for the diet may include information such as personal information, a medical history, tastes and hobbies, family relationships, exercise, supplements, and meals. The information acquisition unit 30 can acquire these various kinds of information.

<Information Output Unit>

The information output unit 32 comprises a plan information output unit 32A, a delivery information output unit 32B, and a monitoring information output unit 32C. The plan information output unit 32A outputs the diet plan created by using the planning unit 34. The target person terminal device 24 shown in FIG. 1 can acquire the diet plan output from the plan information output unit 32A.

The delivery information output unit 32B outputs information on a delivery destination of a blood inspection kit used for the blood inspection of the target person based on the diet plan created by using the planning unit 34. The inspector terminal device 26 shown in FIG. 1 acquires the information of the delivery destination of the blood inspection kit output from the delivery information output unit 32B.

A blood inspection institution (not shown) delivers the blood inspection kit based on the information of the delivery destination of the blood inspection kit. Examples of the delivery destination of the blood inspection kit include home of the target person and a designated store. The store mentioned herein may be a pharmacy, a store other than the pharmacy, and a sales office of a delivery company as long as the target person can receive the blood inspection kit in the store. The blood inspection kit may be delivered from a blood inspection kit providing institution other than the blood inspection institution.

The monitoring information output unit 32C outputs monitoring information indicating a monitoring result of the diet plan implemented by using the monitoring unit 38. The target person terminal device 24 can acquire the monitoring information output from the monitoring information output unit 32C.

Examples of the monitoring information include progress status information indicating a progress status of the diet plan, change proposal information indicating a change proposal of the diet plan, and encouragement information for encouraging the input of the third physical information. The monitoring information output unit 32C corresponds to an example of a progress status information output unit. The monitoring information output unit 32C corresponds to an example of a change proposal information output unit. The monitoring information output unit 32C corresponds to an example of an encouragement information output unit.

<Planning Unit>

The planning unit 34 acquires the physical information via the physical information acquisition unit 30A. The physical information is a general term for the first physical information, the second physical information, and the third physical information. The planning unit 34 acquires the blood inspection item value via the blood inspection item value acquisition unit 30B. The planning unit 34 creates the diet plan based on the physical information and the blood inspection item value. The planning unit 34 stores the diet plan for each target person in a storage unit (not shown).

The diet plan prescribes at least one of the second physical information, an implementation period, the measurement frequency of the third physical information, or the implementation frequency of the blood inspection. The measurement frequency of the third physical information can be prescribed by using the number of times the third physical information is input and the input frequency of the third physical information. The implementation frequency of the blood inspection can be prescribed by using the number of times of the blood inspection and the frequency of the blood inspection.

The number of times of the third physical information is input and the input frequency of the third physical information may be prescribed as one or more input dates of the third physical information. The number of times of the blood inspection and the frequency of the blood inspection may be prescribed as two or more blood inspection dates.

The planning unit 34 outputs the diet plan to the target person terminal device 24 via the plan information output unit 32A. The planning unit 34 outputs the delivery information of the blood inspection kit corresponding to the diet plan for each target person to a delivery source of the blood inspection kit via the delivery information output unit 32B. The delivery information of the blood inspection kit corresponds to the blood inspection kit delivery information.

<Analysis Unit>

The analysis unit 36 analyzes the third physical information acquired by using the physical information acquisition unit 30A and the third blood inspection item value acquired by using the blood inspection item value acquisition unit 30B, and generates the progress status information indicating the progress status of the diet plan. Examples of the progress status information include information indicating that the progress status is ahead of the plan, information indicating that the progress status is proceeding according to the plan, and information indicating that the progress status is behind the plan.

The analysis unit 36 creates the change proposal information indicating the change proposal of the diet plan according to the progress status information. The progress status information and the change proposal information generated by using the analysis unit 36 are output to the monitoring unit 38.

<Monitoring Unit>

The monitoring unit 38 monitors the diet plan by using at least one of an acquisition status of the third physical information or an acquisition status of the blood inspection result. In a case where a delay occurs in the input of the third physical information performed by the target person, the monitoring unit 38 outputs warning information by using the monitoring information output unit 32C. In a case where a delay occurs in the blood inspection performed by the target person, the monitoring unit 38 outputs the warning information by using the monitoring information output unit 32C. Details of the warning information and the output of the warning information will be described later.

[Example of Configuration of Personal Information Database]

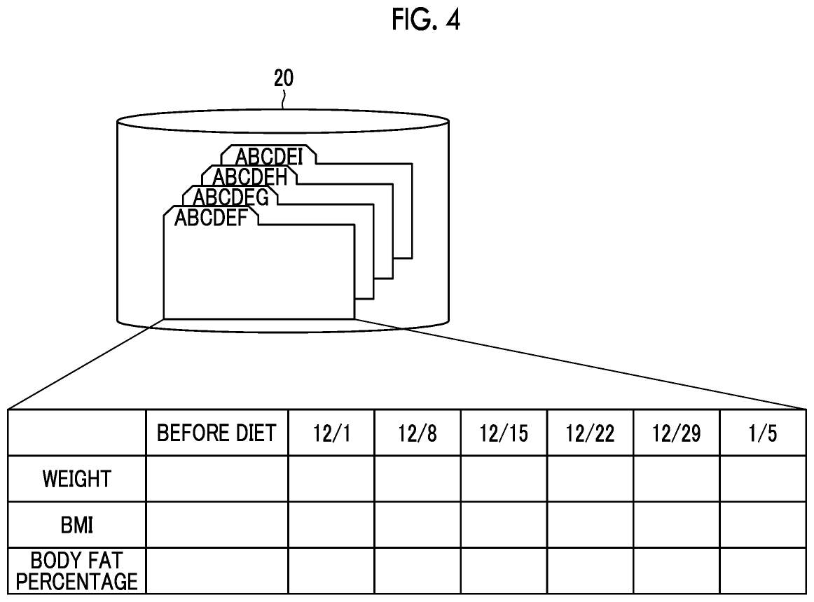

FIG. 4 is a schematic diagram showing a configuration example of the personal information database. The personal information database 20 stores the third physical information for each target person. The personal information database 20 may store the first physical information that is initial physical information.

FIG. 4 shows a case where the weight, the BMI value, and the body fat percentage before the diet are stored as the first physical information and the weight, the BMI value, and the body fat percentage for each measurement day are stored as the third physical information.

The target person transmits the third physical information measured regularly to the personal information database 20 by using the target person terminal device 24. In a case where the first physical information of the target person is stored in the personal information database 20, the server device 12 may read the first physical information from the personal information database 20.

The personal information database 20 stores the third physical information for each target person which is transmitted from the target person terminal device 24. FIG. 4 shows a case where measured values of the weight, the BMI value, and the body fat percentage are input every other week. A blood pressure which may be measured by the target person and may serve as an index of the diet may be added to the third physical information.

The personal information database 20 may store the blood inspection item value of the target person in association with the blood inspection date. The personal information database 20 may store the blood inspection item value of the target person before the diet application. A text string using alphabets shown in FIG. 4 indicates identification information of the target person such as a name and a personal number of the target person.

[Acquisition Example of Blood Inspection Item Value Using Big Database]

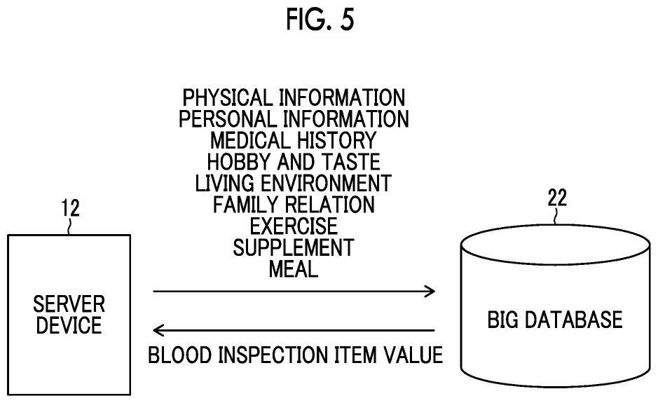

FIG. 5 is a conceptual diagram of blood inspection item value acquisition using the big database. Big data is a huge data group that is difficult to be recorded and stored in a database management system of the related art. In the present embodiment, the big data capable of reading a first blood inspection item value corresponding to the first physical information of the target person and a second blood inspection item value corresponding to the second physical information is applied.

The big data applied to the present embodiment may have, as parameters, information such as personal information, a medical history, hobby and taste information for each year, living environment information, family information, exercise, supplement intake, and meals. Examples of the personal information include a date of birth, an age, a gender, a birthplace, relative information, and personality.

The medical history includes an illness history, a hospital visiting history, a hospitalization history, and a surgery history. Examples of the information on the hobbies and tastes for each year include a smoking status and a drinking status. Examples of the living environment include a place of residence, a climate of the place of residence, regional characteristics of the place of residence, a commuting period, means of commuting, presence or absence of a cohabitant, and friends.

Examples of the family information include information regarding disease histories of relatives and genetics. Examples of the exercise include a kind of the exercise, the number of times of the exercise per week, an exercise period per exercise, and the frequency of the exercise.

Examples of the supplement intake include a kind of the supplement, an intake amount of the supplement, the intake frequency of the supplement, an intake period of the supplement, and an intake time of the supplement. Examples of the meals include a calorie intake per day, nutritional balance, the number of times of the meal per day, and the frequency of the meal.

The server device 12 reads out the blood inspection item value of the target person corresponding to the physical information of the target person by referring to the big database 22. For example, in a case where the initial weight of the target person is 70 kilograms and the target weight is 60 kilograms, the server device 12 reads out the blood inspection item value corresponding to 70 kilograms and the blood inspection item value corresponding to 60 kilograms from the big database 22.

That is, the server device 12 acquires an initial blood inspection item value of the target person and a blood inspection item value corresponding to a target state, and creates the diet plan based on a relationship between these blood inspection item values by using the planning unit 34.

[Procedure of Diet Plan Management Method]

<Overall Flow>

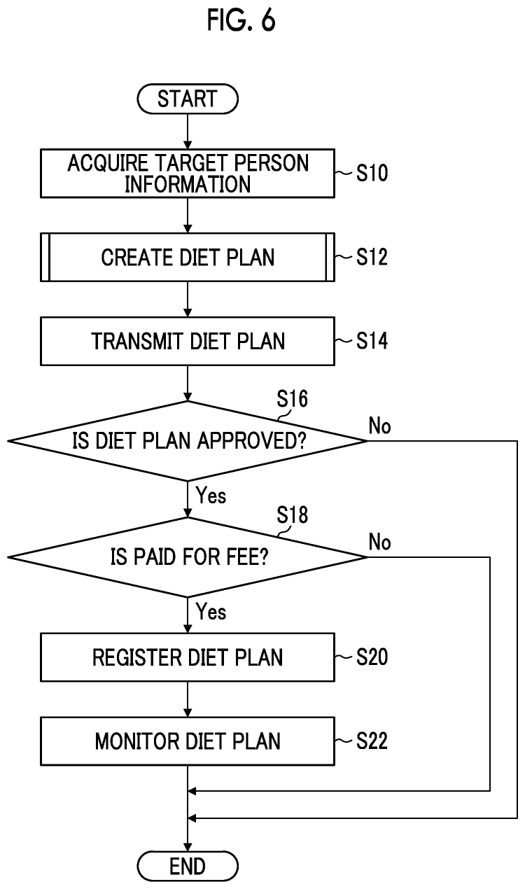

FIG. 6 is a flowchart showing a procedure flow of a diet plan management method according to the first embodiment. In target person information acquisition step S10, the planning unit 34 shown in FIG. 3 acquires the first physical information via the physical information acquisition unit 30A.

In target person information acquisition step S10, the planning unit 34 acquires the first blood inspection item value via the blood inspection item value acquisition unit 30B. The first blood inspection item value stored in advance may be read out, or may be acquired by implementing the blood inspection. After target person information acquisition step S10, the processing proceeds to diet plan creation step S12.

In diet plan creation step S12, the planning unit 34 decides the second physical information. The second physical information may be transmitted from the target person terminal device 24, or may be prescribed. The planning unit 34 creates the diet plan of the target person based on the first physical information, the second physical information, the first blood inspection item value, and the second blood inspection item value. After diet plan creation step S12, the processing proceeds to diet plan transmission step S14.

In diet plan transmission step S14, the planning unit 34 transmits the diet plan to the target person terminal device 24 via the plan information output unit 32A. The planning unit 34 may transmit information indicating that the diet plan is created to the target person terminal device 24. For example, the planning unit 34 can transmit the information indicating that the diet plan is created to the target person terminal device 24 by using an electronic mail and an alert.

The planning unit 34 may transmit an electronic file in which the diet plan is visualized to the target person terminal device 24. The electronic file in which the diet plan is visualized may be downloaded by the target person terminal device 24 according to a download request transmitted from the target person terminal device 24. After diet plan transmission step S14, the processing proceeds to diet plan approval determination step S16.

In diet plan approval determination step S16, the monitoring unit 38 determines whether or not approval information indicating that the diet plan transmitted from the target person terminal device 24 is approved is acquired until a prescribed deadline.

In a case where the monitoring unit 38 does not acquire the approval information until the prescribed deadline in diet plan approval determination step S16, the determination result is No. In a case where the determination result is No, the server device 12 ends the diet plan management method.

Meanwhile, in a case where the monitoring unit 38 acquires the approval information until the prescribed deadline in diet plan approval determination step S16, the determination result is Yes. In a case where the determination result is Yes, the processing proceeds to fee payment determination step S18.

In fee payment determination step S18, the monitoring unit 38 determines whether or not the fee is paid until the prescribed deadline. In a case where the monitoring unit 38 cannot confirm the payment of the fee until the prescribed deadline in fee payment determination step S18, the determination result is No. In a case where the determination result is No, the server device 12 ends the diet plan management method.

Meanwhile, in a case where the monitoring unit 38 confirms the payment of the fee until the prescribed deadline, the determination result is Yes. In a case where the determination result is Yes, the processing proceeds to diet plan registration step S20. In diet plan registration step S20, the monitoring unit 38 registers the target person and the diet plan to be monitored in association with each other. After diet plan registration step S20, the processing proceeds to diet plan monitoring step S22.

In diet plan monitoring step S22, the monitoring unit 38 monitors the diet plan based on the registered diet plan. After diet plan monitoring step S22, the monitoring unit 38 ends the diet plan management method.

<Procedure of Diet Plan Creation Step>

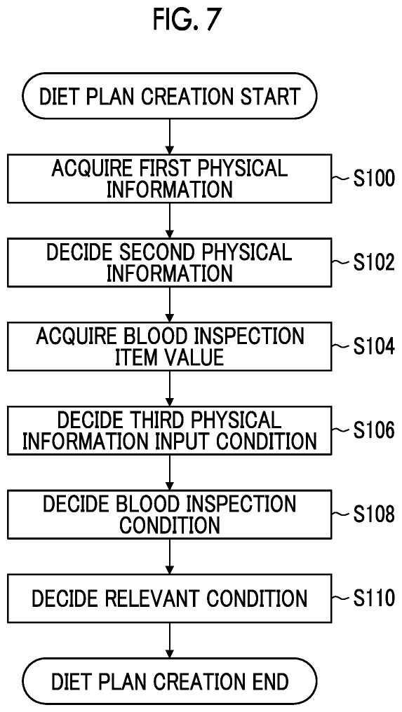

FIG. 7 is a flowchart showing a procedure flow of the diet plan creation step shown in FIG. 6. In the diet plan creation step, first, first physical information acquisition step S100 is executed. In first physical information acquisition step S100, the planning unit 34 acquires the first physical information. After first physical information acquisition step S100, the processing proceeds to second physical information decision step S102.

In second physical information decision step S102, the planning unit 34 decides the second physical information. The planning unit 34 can adopt the second physical information which is transmitted from the target person terminal device 24 and is set by the target person. The planning unit 34 can adopt the prescribed second physical information.

For example, in a case where the diet period is six months, the weight is reduced by ten kilograms, and in a case where the diet period is three months, the weight is reduced by five kilograms. The second physical information is prescribed according to the diet period in some cases. After second physical information decision step S102, the processing proceeds to blood inspection item value acquisition step S104.

In blood inspection item value acquisition step S104, the planning unit 34 acquires the first blood inspection item value and the second blood inspection item value by referring to the big database 22.

For example, in a case where the first physical information is a weight of 70 kilograms, the blood inspection item value corresponding to the weight of 70 kilograms is acquired as the first blood inspection item value. In a case where the second physical information is a weight of 60 kilograms, the blood inspection item value corresponding to the weight of 60 kilograms is acquired as the second blood inspection item value. After blood inspection item value acquisition step S104, the processing proceeds to third physical information input condition decision step S106.

In third physical information input condition decision step S106, the planning unit 34 decides, as a third physical information input condition, the number of times the third physical information is input and the input frequency of the third physical information. The third physical information input condition may be automatically decided from the diet period. For example, in a case where the diet period is six months, the number of times of the input can be decided as 24 and the input frequency can be decided as once a week. The input of the third physical information mentioned herein may be replaced with the measurement of the third physical information.

The third physical information input condition may be decided based on information input by the target person via the target person terminal device 24. That is, the planning unit 34 can decide, as the third physical information input condition, the number of times the third physical information is input by the target person via the target person terminal device 24 and the input frequency of the third physical information. After third physical information input condition decision step S106, the processing proceeds to blood inspection condition decision step S108.

In blood inspection condition decision step S108, the planning unit 34 decides the blood inspection condition. The blood inspection condition includes the number of times of the blood inspection and the frequency of the blood inspection. The number of times of the blood inspection and the frequency of the blood inspection may be decided as an implementation date of the blood inspection.

The blood inspection condition can be decided according to the third physical information input condition. For example, in a case where the diet period is six months, the planning unit 34 can decide that the number of times of the blood inspection is six and the frequency of the blood inspection is once a month. The planning unit 34 can decide, as the blood inspection condition, the number of times of the blood inspection input by the target person via the target person terminal device 24 and the frequency of the blood inspection. After blood inspection condition decision step S108, the processing proceeds to relevant condition decision step S110.

In relevant condition decision step S110, the planning unit 34 decides a relevant condition such as a meal condition, an exercise condition, and a supplement intake condition. Examples of the meal condition include a calorie intake per day, a period between the meals, nutritional balance, and prohibited food. For example, a drinking condition such as one beer having a drinking capacity of 350 ml per day, drinking allowed five days a week, and drinking prohibited two days a week can be decided as the meal condition.

Examples of the exercise condition include the frequency of the exercise, one exercise period, and calorie consumption. For example, walking for 30 minutes or more every day can be decided as the exercise condition. Examples of the supplement include supplements to be taken, the intake amount, and the intake frequency. After relevant condition decision step S110, the planning unit 34 ends the diet plan creation step.

<Procedure of Diet Plan Monitoring Step>

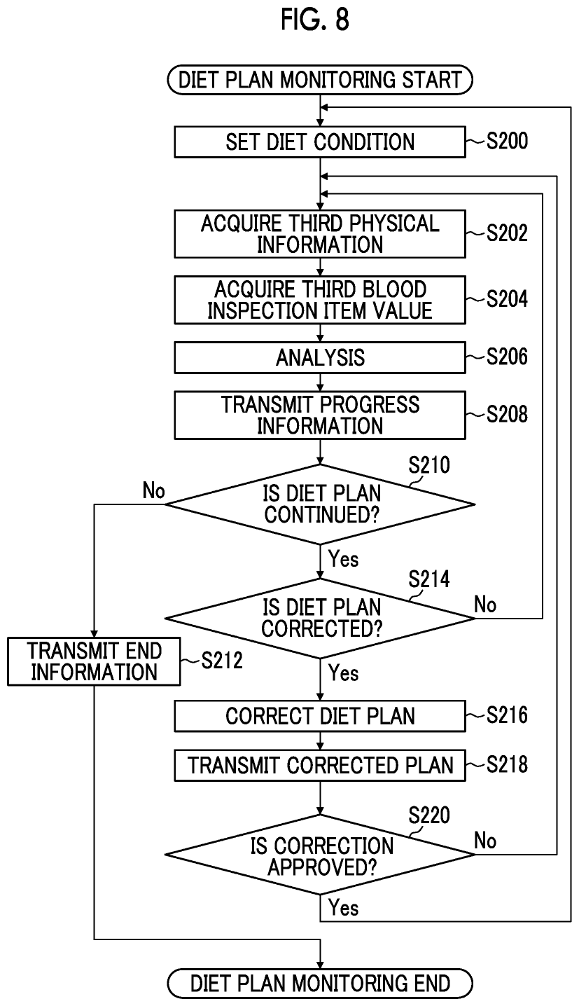

FIG. 8 is a flowchart showing a procedure flow of the diet plan monitoring step shown in FIG. 6. In the diet plan monitoring step, first, diet condition setting step S200 is executed. In diet condition setting step S200, the monitoring unit 38 reads out the diet plan registered in diet plan registration step S20 shown in FIG. 6, and sets a prescribed diet condition.

The diet condition set in diet condition setting step S200 includes the third physical information input condition and the blood inspection condition. After diet condition setting step S200, the processing proceeds to third physical information acquisition step S202.

In third physical information acquisition step S202, the monitoring unit 38 acquires the latest third physical information of the target person from the personal information database 20 according to the third physical information input condition. The latest third physical information is the third physical information last stored in the personal information database 20. After third physical information acquisition step S202, the processing proceeds to third blood inspection item value acquisition step S204.

In third blood inspection item value acquisition step S204, the monitoring unit 38 acquires the latest third blood inspection item value of the target person from the storage device that stores the blood inspection result according to the blood inspection condition. The latest third blood inspection item value is the third blood inspection item value last stored.

In third blood inspection item value acquisition step S204, the monitoring unit 38 may acquire all the third blood inspection item values acquired as the result of the blood inspection, or may selectively acquire a part of the third blood inspection items acquired as the result of the blood inspection. After third blood inspection item value acquisition step S204, the processing proceeds to analysis step S206.

In analysis step S206, the analysis unit 36 analyzes the diet plan by using the latest third physical information and the latest third blood inspection item value. For example, the analysis unit 36 analyzes whether or not the third physical information is changing according to the plan and whether or not the third blood inspection item value is changing according to the plan.

In a case where at least one of the third physical information or the third blood inspection item value is not changed according to the plan, the analysis unit 36 analyzes whether or not the diet plan can be changed. Examples of the change of the diet plan include change of the diet period and change of the second physical information. The analysis result of the diet plan using the analysis unit 36 is transmitted to the monitoring unit 38. After analysis step S206, the processing proceeds to progress status information transmission step S208.

In progress status information transmission step S208, the monitoring unit 38 transmits at least one of progress status information indicating the progress of the diet plan or result information indicating the result of the diet plan to the target person terminal device 24 via the monitoring information output unit 32C.

The target person terminal device 24 can receive the progress status information and the result information transmitted from the monitoring unit 38. In progress status information transmission step S208, the monitoring unit 38 may transmit the progress status information or notification information indicating that the result information is stored to the target person terminal device 24.

In a case where the target person terminal device 24 receives the notification information, the target person may access the storage device that stores the progress status information or the result information by using the target person terminal device 24, and may acquire the progress status information or the result information. After progress status information transmission step S208, the processing proceeds to diet plan continuation determination step S210.

In diet plan continuation determination step S210, the monitoring unit 38 determines whether or not continuation confirmation information indicating whether or not to continue the diet plan or end the diet plan is received.

That is, the target person who acquires the progress status information of the diet plan can transmit the continuation confirmation information indicating whether or not to continue the diet plan or end the diet plan to the server device 12 by using the target person terminal device 24.

In a case where the monitoring unit 38 receives the continuation confirmation information indicating that the diet plan is ended in diet plan continuation determination step S210, the determination result is No. In a case where the acquisition of the third physical information, the acquisition of the third blood inspection item value, and the analysis as much as the number of times prescribed in the diet plan are ended, the determination result is also No. In a case where the determination result is No, the processing proceeds to end information transmission step S212.

In end information transmission step S212, the monitoring unit 38 transmits end information indicating that the diet plan is ended to the target person terminal device 24. After end information transmission step S212, the monitoring unit 38 ends the diet plan monitoring step.

Meanwhile, in a case where the monitoring unit 38 receives the continuation confirmation information indicating that the diet plan is continued in diet plan continuation determination step S210, the determination result is Yes. In a case where the monitoring unit 38 does not receive the continuation confirmation information, the determination result is Yes. In a case where the determination result is Yes, the processing proceeds to diet plan correction determination step S214.

In diet plan correction determination step S214, the monitoring unit 38 determines whether or not correction information indicating that the diet plan transmitted from the target person terminal device 24 is corrected is received. That is, in a case where the target person desires to continue the diet plan and desires to change the diet plan, the target person can transmit information indicating such intention of the target person to the server device 12 by using the target person terminal device 24.

In a case where the monitoring unit 38 does not receive the correction information in diet plan correction determination step S214, the determination result is No. In a case where the determination result is No, the processing proceeds to third physical information acquisition step S202, and steps from third physical information acquisition step S202 to diet plan correction determination step S214 are executed until the determination result becomes No in diet plan continuation determination step S210.

Meanwhile, in a case where the monitoring unit 38 receives the correction information in diet plan correction determination step S214, the determination result is Yes. In a case where the determination result is Yes, the processing proceeds to diet plan correction step S216.

In diet plan correction step S216, the planning unit 34 corrects the diet plan. Examples of the correction of the diet plan include correction of the diet period and correction of the second physical information. After diet plan correction step S216, the processing proceeds to correction plan transmission step S218. In correction plan transmission step S218, the planning unit 34 transmits the corrected diet plan to the target person terminal device 24.

The target person can confirm the corrected diet plan transmitted from the server device 12 by using the target person terminal device 24. The target person can transmit information indicating that the corrected diet plan is approved to the server device 12 by using the target person terminal device 24. After correction plan transmission step S218, the processing proceeds to correction approval determination step S220.

In correction approval determination step S220, the monitoring unit 38 determines whether or not approval information indicating that the correction of the diet plan is approved is received from the target person terminal device 24. In a case where the monitoring unit 38 does not receive the approval information within the prescribed period in correction approval determination step S220, the determination result is No. In a case where the determination result is No, the processing proceeds to third physical information acquisition step S202, and steps from third physical information acquisition step S202 to correction approval determination step S220 are executed until the determination result becomes No in diet plan continuation determination step S210.

Meanwhile, in a case where the monitoring unit 38 receives the approval information within the prescribed period in correction approval determination step S220, the determination result is Yes. In a case where the determination result is Yes, the processing proceeds to diet condition setting step S200, and the diet condition prescribed in the corrected diet plan is set.

Hereinafter, the steps from diet condition setting step S200 to diet plan correction step S216 are repeatedly executed until the determination result is No in diet plan continuation determination step S210.

<Procedure of Third Physical Information Acquisition Step>

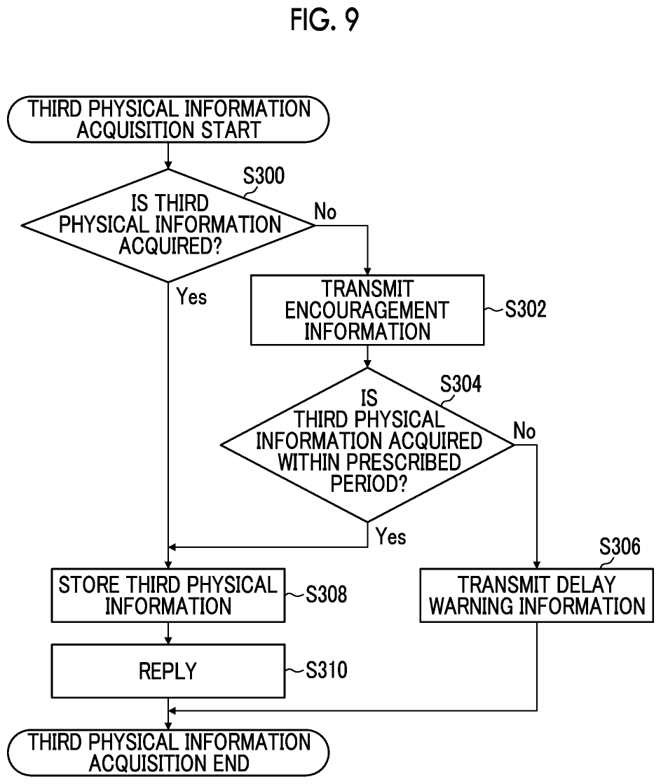

FIG. 9 is a flowchart showing a procedure flow of the third physical information acquisition step shown in FIG. 8. In the weight information acquisition step, first, acquisition confirmation step S300 is executed. In acquisition confirmation step S300, the monitoring unit 38 determines whether or not the third physical information is acquired at the prescribed acquisition frequency from the personal information database 20. That is, the monitoring unit 38 determines whether or not the third physical information of the target person is input during a period from a date at which the third physical information is input to a date before a date at which the next third physical information is input.

In a case where the third physical information is not input during the period from the date at which the third physical information is input to the date before the date at which the next third physical information is input in acquisition confirmation step S300, the determination result is No. In a case where the determination is No, the processing proceeds to encouragement information transmission step S302.

In encouragement information transmission step S302, the monitoring unit 38 transmits the encouragement information for encouraging the target person terminal device 24 to input the third physical information. After encouragement information transmission step S302, the processing proceeds to prescribed period elapse determination step S304.

In prescribed period elapse determination step S304, the monitoring unit 38 determines whether or not the third physical information is acquired within the prescribed period from the encouragement information transmission. In a case where the third physical information is not acquired within the prescribed period in prescribed period elapse determination step S304, the determination result is No. In a case where the determination result is No, the processing proceeds to delay warning information transmission step S306.

In delay warning information transmission step S306, the monitoring unit 38 transmits delay warning information indicating that the input of the third physical information is delayed to the target person terminal device 24. The target person terminal device 24 can receive the delay warning information transmitted from the monitoring unit 38. After delay warning information transmission step S306, the monitoring unit 38 ends the third physical information acquisition step.

Meanwhile, in a case where the third physical information is acquired within the prescribed period from the encouragement information transmission in prescribed period elapse determination step S304, the determination result is Yes. In a case where the determination result is Yes, the processing proceeds to third physical information storing step S308.

In a case where the third physical information is acquired during the period from the date at which the third physical information is input to a date before the date at which the next third physical information is input in acquisition confirmation step S300, the determination result is Yes. In a case where the determination result is Yes, the processing proceeds to third physical information storing step S308.

In third physical information storing step S308, the monitoring unit 38 stores the third physical information acquired from the personal information database 20. After third physical information storing step S308, the processing proceeds to reply step S310.

In reply step S310, the monitoring unit 38 transmits reply information indicating that the third physical information is input during the period from the date at which the third physical information is input to the date before the date at which the next third physical information is input to the target person terminal device 24. After reply step S310, the monitoring unit 38 ends the third physical information acquisition step.

<Procedure of Third Blood Inspection Item Value Acquisition Step>

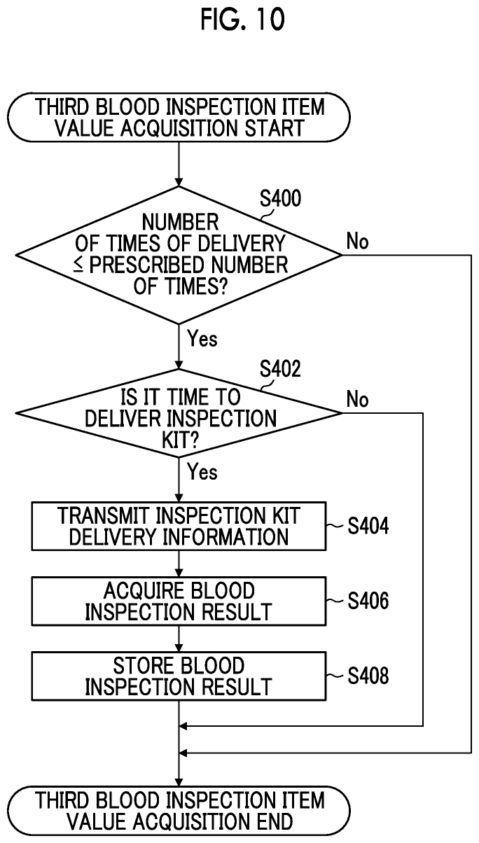

FIG. 10 is a flowchart showing a procedure flow of the third blood inspection item value acquisition step shown in FIG. 8. In the third blood inspection item value acquisition step, first, delivery number-of-times confirmation step S400 is executed. In delivery number-of-times confirmation step S400, the monitoring unit 38 determines whether or not the number of times the blood inspection kit is delivered is less than a prescribed number of times. The number of times the blood inspection kit is delivered may be replaced with the number of times of the blood inspection.

In a case where the monitoring unit 38 determines that the number of times the blood inspection kit is delivered exceeds the prescribed number of times in delivery number-of-times confirmation step S400, the determination result is No. In a case where the determination result is No, the monitoring unit 38 ends the third blood inspection item value acquisition step.

Meanwhile, in a case where the monitoring unit 38 determines that the number of times the blood inspection kit is delivered is equal to or less than the prescribed number of times in delivery number-of-times confirmation step S400, the determination result is Yes. In a case where the determination result is Yes, the processing proceeds to delivery timing determination step S402.

In delivery timing determination step S402, the monitoring unit 38 determines whether or not it is time to deliver the blood inspection kit. In a case where the monitoring unit 38 determines that it is not time to deliver the blood inspection kit in delivery timing determination step S402, the determination result is No. In a case where the determination result is No, the monitoring unit 38 ends the third blood inspection item value acquisition step.

Meanwhile, in a case where the monitoring unit 38 determines that it is time to deliver the blood inspection kit in delivery timing determination step S402, the determination result is Yes. In a case where the determination result is Yes, the processing proceeds to inspection kit delivery information transmission step S404.

In inspection kit delivery information transmission step S404, the planning unit 34 transmits the delivery information of the blood inspection kit to the delivery source of the blood inspection kit. The delivery source of the blood inspection kit transmits the blood inspection kit to a prescribed delivery destination based on the delivery information of the blood inspection kit.

The delivery information of the blood inspection kit to the delivery source of the blood inspection kit may be transmitted via the blood inspection institution, or may be transmitted via a distributor of the blood inspection kit. After inspection kit delivery information transmission step S404, the processing proceeds to inspection result acquisition step S406.

In inspection result acquisition step S406, the blood inspection item value acquisition unit 30B acquires the blood inspection result. The blood inspection result mentioned herein includes at least the third blood inspection item value required in a case where the progress of the diet plan is analyzed. After inspection result acquisition step S406, the processing proceeds to inspection result storing step S408.

In inspection result storing step S408, the blood inspection item value acquisition unit 30B stores the blood inspection result. In inspection result storing step S408, only the third blood inspection item value necessary in a case where the progress of the diet plan is analyzed may be stored. After the blood inspection result is stored in inspection result storing step S408, the monitoring unit 38 ends the blood inspection item value acquisition step.

In the blood inspection item value acquisition step shown in FIG. 10, in a case where the blood inspection result acquisition is delayed, the blood inspection encouragement information for encouraging the target person terminal device 24 or the blood inspection institution to implement the blood inspection may be transmitted.

<Flow of Information Between Components of Diet Plan Management System and Flow of Blood Inspection Kit>

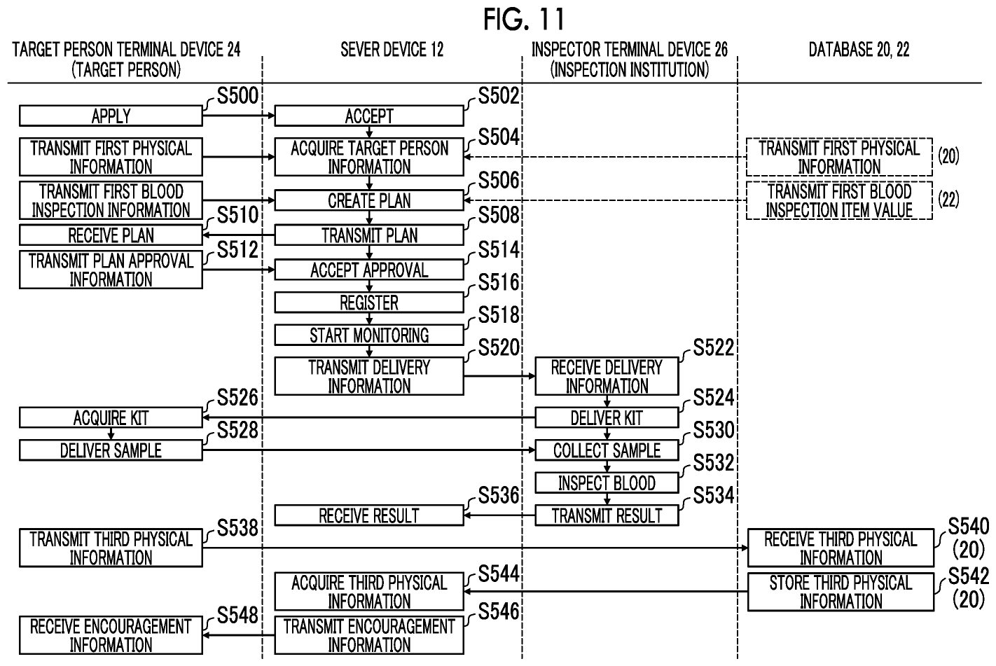

FIG. 11 is a flowchart showing a flow of information between the components of the diet plan management system and a flow of the blood inspection kit. A reference 20 shown in FIG. 11 indicates the personal information database 20 shown in FIG. 1. A reference 22 indicates the big database 22 shown in FIG. 1.

In application step S500, diet application information is transmitted from the target person terminal device 24 to the server device 12. In acceptance step S502, the server device 12 accepts a diet application transmitted from the target person terminal device 24.

In target person information acquisition step S504, information of the target person who applies for the diet is acquired. Target person information acquisition step S504 corresponds to target person information acquisition step S10 shown in FIG. 6. In target person information acquisition step S504, the server device 12 acquires the first physical information of the target person from the personal information database 20. In a case where the first physical information of the target person is not stored in the personal information database 20, the first physical information of the target person may be acquired from the target person terminal device 24.

In diet plan creation step S506, the server device 12 acquires the second blood inspection item value transmitted from the big database 22. In diet plan creation step S506, the first blood inspection item value is acquired. In a case where the target person has the blood inspection result, the first blood inspection item value can be acquired from the target person terminal device 24. In a case where the target person does not have the blood inspection result, the first blood inspection item value can be acquired from the inspector terminal device 26.

In diet plan creation step S506, the diet plan of the target person is created based on the first physical information, the second physical information, the first blood inspection item value, and the second blood inspection item value. In diet plan transmission step S508, the server device 12 transmits the diet plan to the target person terminal device 24. Diet plan transmission step S508 corresponds to diet plan transmission step S14 shown in FIG. 6.

In diet plan reception step S510, the target person terminal device 24 receives the diet plan transmitted from the server device 12. In diet plan approval transmission step S512, the approval information indicating that the diet plan is approved is transmitted from the target person terminal device 24 to the server device 12.

In diet plan approval reception step S514, the server device 12 receives the approval information transmitted from the target person terminal device 24. Diet plan approval reception step S514 corresponds to diet plan approval determination step S16.

In registration step S516, the server device 12 registers the approved diet plan. Registration step S516 corresponds to diet plan registration step S20 shown in FIG. 6. In monitoring start step S518, the server device 12 starts monitoring the diet plan. Monitoring start step S518 is a component of diet plan monitoring step S22 shown in FIG. 6.

In delivery information transmission step S520, the server device 12 transmits the delivery information of the blood inspection kit to the delivery source of the blood inspection kit. In delivery information reception step S522, the inspector terminal device 26 receives the delivery information of the blood inspection kit transmitted from the server device 12. In the example shown in FIG. 11, the delivery source of the blood inspection kit is the blood inspection institution.

In kit delivery step S524, the blood inspection institution transmits the blood inspection kit to the target person. In kit acquisition step S526, the target person acquires the blood inspection kit delivered from the blood inspection institution. In sample delivery step S528, the target person delivers blood which is a sample of the target person collected by using the blood inspection kit to the blood inspection institution.

In sample collection step S530, the blood inspection institution collects the sample of the target person delivered from the target person. In inspection step S532, the blood inspection institution inspects the sample of the target person. In result transmission step S534, the inspector terminal device 26 transmits the blood inspection result of the target person to the server device 12.

In result reception step S536, the server device 12 receives the blood inspection result of the target person transmitted from the inspector terminal device 26. In a case where a delay occurs in the blood inspection result acquisition, the server device 12 may transmit the encouragement information for encouraging the implementation of the blood inspection to at least one of the target person terminal device 24 or the inspector terminal device 26.

In third physical information transmission step S538, the target person terminal device 24 transmits the third physical information measured based on the diet plan to the personal information database 20. In third physical information reception step S540, the personal information database 20 receives the third physical information transmitted from the target person terminal device 24. In third physical information storing step S542, the personal information database 20 stores the received third physical information.

In third physical information acquisition step S544, the server device 12 acquires the third physical information from the personal information database 20 based on the diet plan. Third physical information acquisition step S544 corresponds to third physical information acquisition step S202 shown in FIG. 8.

In encouragement information transmission step S546, the server device 12 transmits, to the target person terminal device 24, the encouragement information for encouraging the measurement of the third physical information and the transmission of the third physical information to the personal information database 20. Encouragement information transmission step S546 corresponds to encouragement information transmission step S302 shown in FIG. 9. In encouragement information reception step S548, the target person terminal device 24 receives the encouragement information transmitted from the server device 12.

[Specific Example of Diet Plan Creation]

Next, a specific example of diet plan creation will be described. First, information exceeding a standard range is extracted from among the first physical information acquired in first physical information acquisition step S100 shown in FIG. 7. Subsequently, the first blood inspection item exceeding a standard range is extracted from among the first blood inspection item values acquired in blood inspection item value acquisition step S104.

Subsequently, the second physical information is set. The second blood inspection item value corresponding to the second physical information is acquired from the big database 22 while referring to the big database 22 shown in FIG. 1 by using the first physical information exceeding the standard range among the first physical information and the first blood inspection item value that exceeds the standard range among the first blood inspection item values. The second blood inspection item value acquired from the big database 22 may include at least the blood inspection item value exceeding a standard range among the first blood inspection item values.

In this manner, the diet plan is created based on the second physical information that is the target physical information and the second blood inspection item value that is the target blood inspection item value.

[Example of Display Screen on Target Person Terminal Device]

The target person terminal device 24 comprises a display device capable of displaying various display screens. In a case where the target person terminal device 24 is a personal computer, a monitor device connected to a computer body is provided. In a case where the target person terminal device 24 is a mobile communication device such as a smartphone, a liquid crystal display is provided in a body of the mobile communication device. The display device corresponds to an example of a display unit.

<Application Screen>

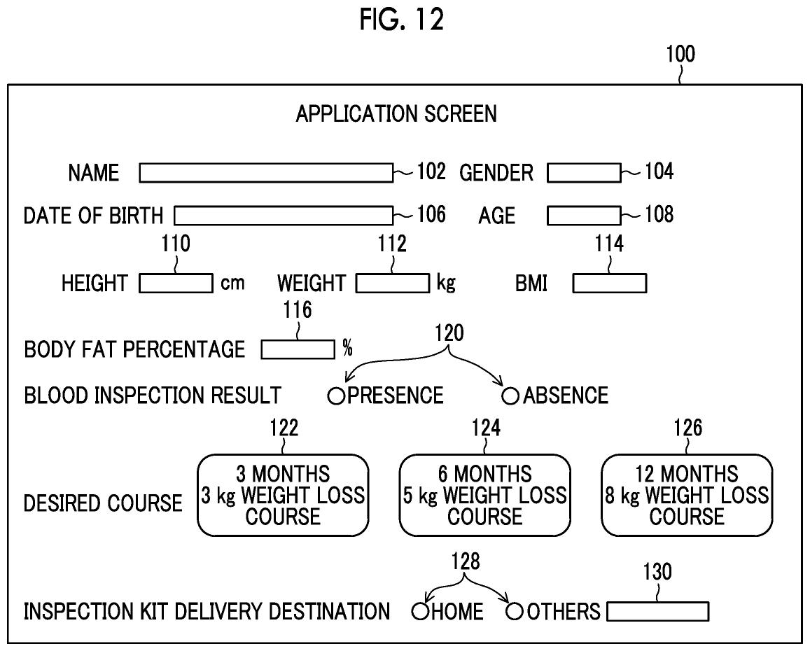

FIG. 12 is an explanatory diagram showing an example of an application screen. An application screen 100 shown in FIG. 12 includes a name input field 102, a gender input field 104, a date of birth input field 106, an age input field 108, a height input field 110, a weight input field 112, a BMI value input field 114, and a body fat percentage input field 116.

A pull-down menu may be applied to each input field. The application screen 100 shown in FIG. 12 is applied in a case where the target person transmits, to the server device 12, the first physical information used in the case of the creation of the diet plan.

The application screen 100 includes a blood inspection result presence and absence selection button 120, a first desired course selection button 122, a second desired course selection button 124, a third desired course selection button 126, a kit delivery destination selection button 128, and a kit delivery destination input field 130.

The target person terminal device 24 shown in FIG. 1 displays the application screen 100 in a case where the target person applies for the diet. The target person can input information necessary in the case of the application for the diet by using operation members such as a keyboard, a mouse, and a touch panel while referring to the application screen 100.

<Diet Plan Proposal Screen>

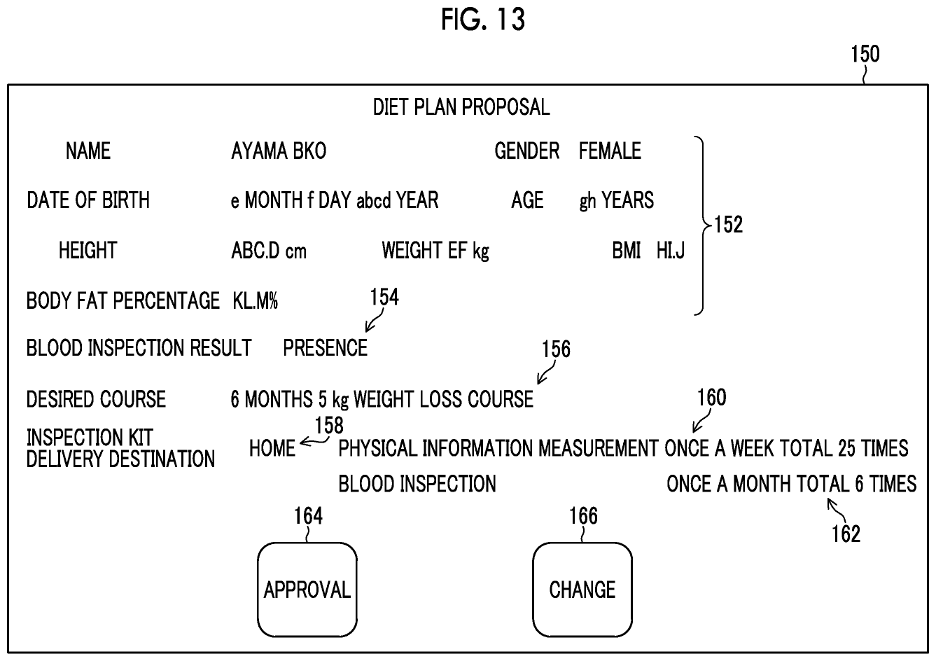

FIG. 13 is an explanatory diagram showing an example of a diet plan proposal screen. A diet plan proposal screen 150 shown in FIG. 13 includes a target person information display field 152, a blood inspection result presence and absence display field 154, a desired course display field 156, a blood inspection kit delivery destination display field 158, and a first physical information measurement condition display field 160, and a blood inspection condition display field 162.

The target person information display field 152 displays target person information such as the name of the target person. Alphabets shown in FIG. 13 as the target person information indicates a character indicating the target person information and a numerical value.

The diet plan proposal screen 150 includes an approval button 164 and a change button 166. In a case where a diet plan proposal displayed on the diet plan proposal screen 150 is approved and the target person clicks the approval button 164, approval information indicating an approval of the diet plan proposal is transmitted from the target person terminal device 24 to the server device 12.

Meanwhile, in a case where the user desires to change the diet plan proposal displayed on the diet plan proposal screen 150 and the target person clicks the change button 166, change desire information indicating that the user desires to change the diet plan proposal is transmitted from the target person terminal device 24 to the server device 12.

The second physical information and the second blood inspection item value may be displayed on the diet plan proposal screen 150. The relevant condition such as the meal condition and the exercise condition may be displayed on the diet plan proposal screen 150. The diet plan proposal screen 150 corresponds to an example of a proposal screen showing the proposal content of the diet plan.

<Diet Acceptance Screen>

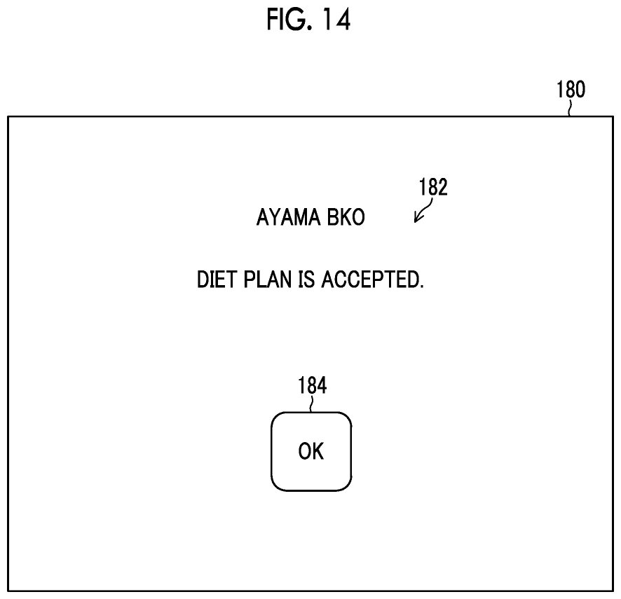

FIG. 14 is an explanatory diagram showing an example of a diet acceptance screen. A diet acceptance screen 180 shown in FIG. 14 includes acceptance completion information 182 indicating that the diet plan transmitted from the server device 12 to the target person terminal device 24 is accepted. The diet acceptance screen 180 includes an OK button 184.

The target person who sees the acceptance completion information 182 on the diet acceptance screen 180 can grasp that the acceptance of the applied diet plan is completed. The target person clicks the OK button 184, and thus, acceptance confirmation information indicating that the target person confirms that the acceptance of the diet plan is completed is transmitted from the target person terminal device 24 to the server device 12.

<Weight Information Input Screen>

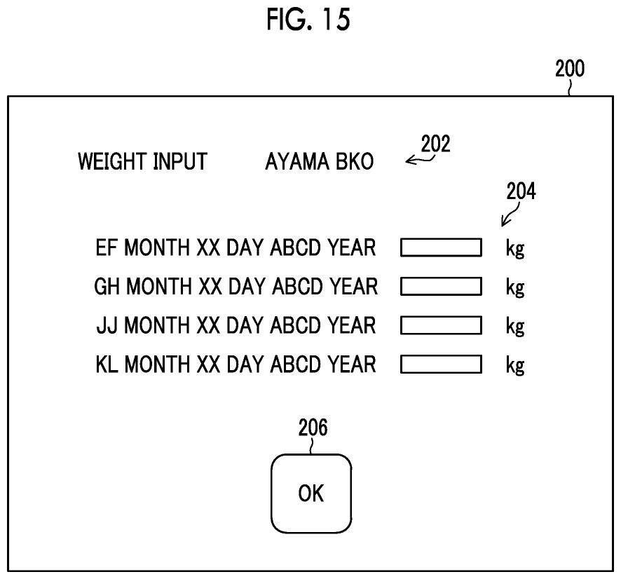

FIG. 15 is an explanatory diagram showing an example of a weight information input screen. FIG. 15 shows a weight information input screen 200 in a case where the weight is applied as the third physical information. Alphabets shown in FIG. 15 as a target person name and a date indicate characters indicating the target person name and numerical values indicating the date.

The weight information input screen 200 shown in FIG. 15 includes a target person name display field 202 and a weight input field 204. FIG. 15 shows four weight input fields 204 for inputting the weight information obtained from weight measurements performed once a week for a total of four times. The target person can input the weight information which is a measurement value for each weight measurement by using an operation member connected to the target person terminal device 24.

An OK button 206 is displayed on the weight information input screen 200. In a case where the target person clicks the OK button 206, the weight information input to the weight input field 204 is transmitted from the target person terminal device 24 to the personal information database 20.

<Progress Report Screen>

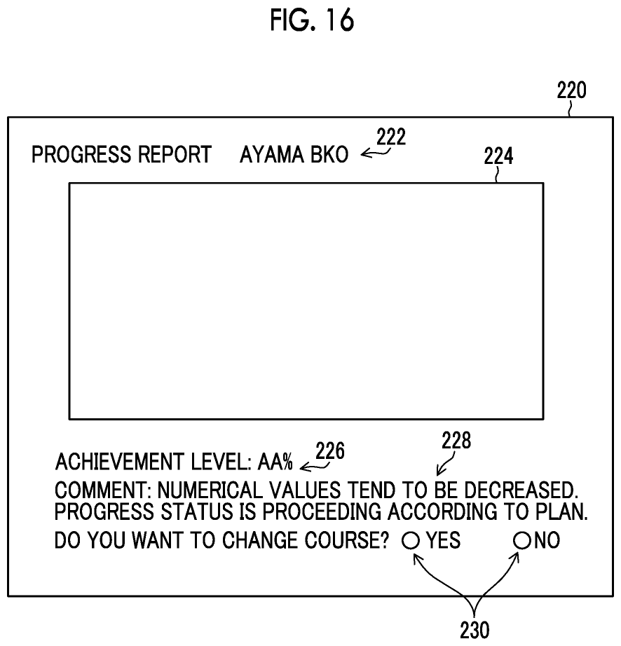

FIG. 16 is an explanatory diagram showing an example of a progress report screen. A target person name display field 222, a blood inspection item value display field 224, an achievement level display field 226, a comment display field 228, and a course change selection button 230 are displayed on a progress report screen 220 shown in FIG. 16.

The target person name display field 222 displays the target person name. The third blood inspection item value obtained from the blood inspection is displayed in the blood inspection item value display field 224. In the blood inspection item value display field 224, all the third blood inspection items included in the blood inspection result may be displayed, or only the third blood inspection item used in a case where the diet plan is created may be displayed.

In the blood inspection item value display field 224, a list indicating the third blood inspection item values for each inspection date may be displayed, or a graph indicating the third blood inspection item values for each inspection date may be displayed. The measurement value of the physical information for each measurement date may be displayed on the progress report screen 220. In FIG. 16, a specific illustration of the blood inspection item value display field 224 is omitted.

An achievement level of the diet plan is displayed in the achievement level display field 226. FIG. 16 shows a case where the achievement level expressed by a percentage is displayed. As an example of the achievement level of the blood inspection item value expressed by the percentage, a value is obtained by subtracting the second blood inspection item value from the current third blood inspection item value, dividing the resultant value of the subtraction by the second blood inspection item value, and multiplying the resultant value of division by 100 is used. The achievement level of the physical information may be calculated, and the achievement level of the blood inspection item value and the achievement level of the physical information may be described together.

A comment regarding the progress of the diet plan is displayed in a comment display field 228. FIG. 16 shows a case where a comment indicating that the diet plan is smoothly proceeding is displayed.

The course change selection button 230 is clicked in a case where the target person selects whether or not to change a diet course. The target person can select whether or not to change the diet course by using the course change selection button 230.

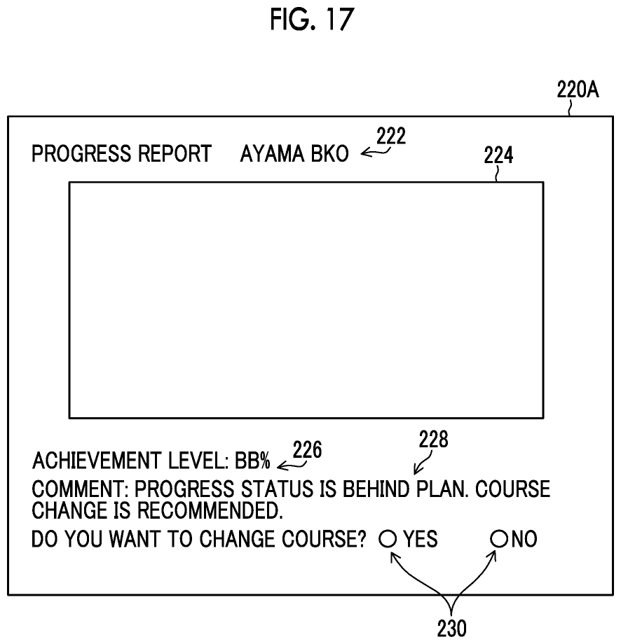

FIG. 17 is an explanatory diagram showing another example of a progress report screen. A progress report screen 220A shown in FIG. 17 is different from the progress report screen 220 shown in FIG. 16 in that the achievement level displayed in the achievement level display field 226 and the contents of the comment displayed in the comment display field 228 are different. That is, in a case where the diet plan is not proceeding according to the plan, the progress report screen 220A shown in FIG. 17 is displayed on the display device of the target person terminal device 24.

The progress report screen 220 and the progress report screen 220A correspond to an example of a progress screen showing the progress of the diet plan.

[Modification Example of First Embodiment]

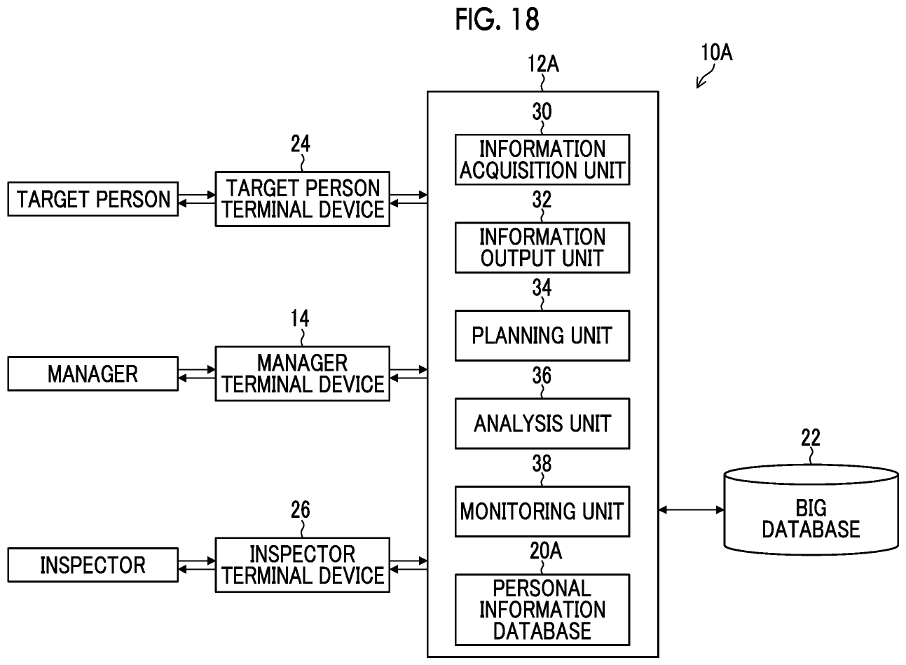

FIG. 18 is a block diagram showing a configuration example of a diet plan management system according to a modification example of the first embodiment. In a diet plan management system 10A shown in FIG. 18, a server device 12A comprises a personal information database 20A. Other components are the same as the components of the diet plan management system 10 shown in FIG. 1, and thus, description thereof will be omitted below.

[Application Example of First Embodiment]

Although it has been described in the present embodiment that the aspect in which the diet plan is created according to the application of the target person who desires the diet, the diet plan may be created for a person who implements the blood inspection regardless of whether or not the persons desires the diet, and the diet plan may be proposed.

The diet plans may be created for all persons who implement the blood inspection, or the diet plan may be created for a part selected from the persons who implement the blood inspection. For example, questionnaires may be attached to blood inspection application forms, and the diet plans may be created for persons who answer the questionnaires and write the physical information.

The diet plans may be created for persons who implement the blood inspection regularly or irregularly by using the blood inspection kits, and the diet plans may be proposed to these persons.

[Operations and Effects of First Embodiment]

According to the diet plan management system 10, the server device 12, and the diet plan management method according to the first embodiment, the following operations and effects can be obtained.

[1]

The diet plan including one or more measurements of the physical information and one or more blood inspections is created by using the first physical information, the second physical information, the first blood inspection item value corresponding to the first physical information, and the second blood inspection item value corresponding to the second physical information. The diet plan is managed based on the third physical information obtained from the measurement of the physical information and the third blood inspection item value obtained from the result of the blood inspection. Accordingly, it is possible to manage the diet plan in which the blood inspection is combined with the measurement of the third physical information.

[2]

The progress of the diet plan can be grasped by using the third blood inspection item value which may be an index of a lifestyle-related disease. Accordingly, the goal of the diet plan is clarified, and thus, motivation to implement the diet plan and continuity of the diet plan implementation are expected to be improved.

For example, even though the weight loss is not achieved according to the plan, in a case where a blood sugar value and a value such as HbA1c are changing according to the plan, the target person can grasp that the diet plan is proceeding according to the plan, and the motivation of the target person is expected to be improved.

[3]

The blood inspection is implemented by using the blood inspection kit. Accordingly, the blood inspection can be implemented without the target person having to go to the medical institution whenever the blood inspection is implemented. The amount of blood collected during the blood inspection may be small, and the blood inspection can be easily implemented.

[4]

The server device 12 displays the progress report screen 220 indicating the progress of the diet plan on the display device of the target person terminal device 24. Accordingly, the target person can grasp the progress of the diet plan by looking at the progress report screen 220. The same applies to the server device 12A.

[5]

The course change selection button 230 is displayed on the progress report screen 220 and the progress report screen 220A. Accordingly, the continuity of the diet plan or the change of the diet plan can be selected.

[6]

At least one item value of blood sugar, HbA1c, HDL cholesterol, LDL cholesterol, neutral fat, AST, ALT, γ-GPT, ALB, ALP, or e-GFR is acquired as the blood inspection item value. Accordingly, the effect of the diet can be grasped according to the change of the blood inspection item value.

### Second Embodiment

[Procedure of Diet Plan Management Method According to Second Embodiment]

Next, a diet plan management method according to a second embodiment will be described. In the diet plan management method according to the second embodiment, the server device 12 transmits drop concern information to the target person terminal device 24.

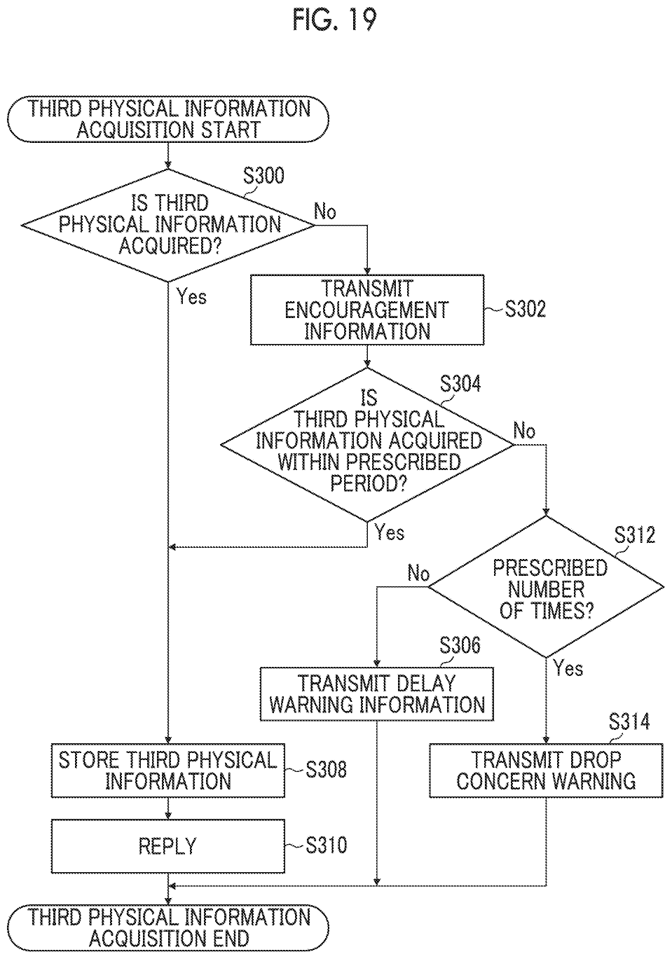

FIG. 19 is a flowchart showing a procedure flow of a third physical information acquisition step in the diet plan management method according to the second embodiment. In the third physical information acquisition step in the diet plan management method according to the second embodiment, number-of-times determination step S312 and drop concern warning transmission step S314 are added to the third physical information acquisition step shown in FIG. 9.

In a case where the determination result is No in prescribed period elapse determination step S304, the processing proceeds to number-of-times determination step S312. In number-of-times determination step S312, the monitoring unit 38 determines whether or not the delay of the first physical information measurement at which the encouragement information is transmitted is equal to or greater than a prescribed number of times.

In a case where the monitoring unit 38 determines in number-of-times determination step S312 that the delay of the third physical information measurement at which the encouragement information is transmitted is less than the prescribed number of times, the determination result is No. In a case where the determination result is No, the processing proceeds to delay warning information transmission step S306.

Meanwhile, in a case where the monitoring unit 38 determines in number-of-times determination step S312 that the delay of the third physical information measurement at which the encouragement information is transmitted is equal to or greater than the prescribed number of times, the determination result is Yes. In a case where the determination result is Yes, the processing proceeds to drop concern warning transmission step S314.

In drop concern warning transmission step S314, the monitoring unit 38 transmits a drop concern warning to the target person terminal device 24. The drop concern warning is warning information indicating that there is a concern of dropping out of the diet plan. After drop concern warning transmission step S314, the monitoring unit 38 ends the third physical information acquisition step.

[Configuration Example of Drop Concern Warning Screen]

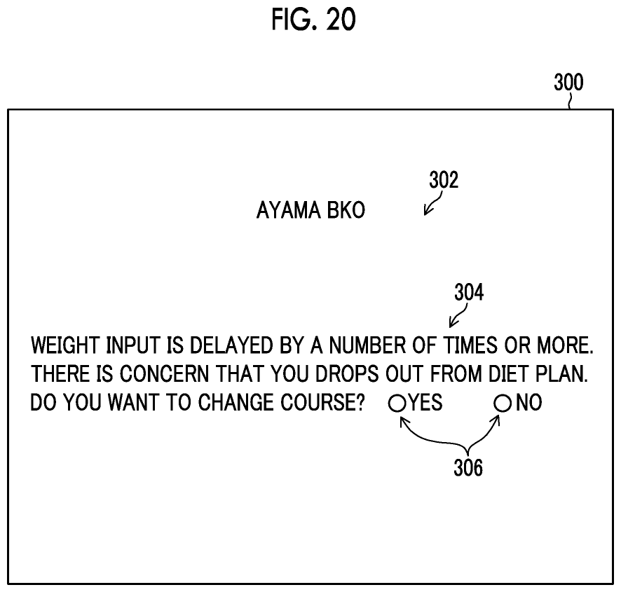

FIG. 20 is an explanatory diagram showing an example of a drop concern warning screen. The drop concern warning screen 300 shown in FIG. 20 includes a target person name display field 302, a drop concern warning display field 304, and a selection button 306.

The drop concern warning screen 300 is displayed by using the display device of the target person terminal device 24. The target person who visually recognizes the drop concern warning screen 300 selects whether or not to change the diet plan from the drop concern warning screen 300 by operating the operation member of the target person terminal device 24. The target person terminal device 24 can transmit selection information for selecting whether or not to change the diet plan to the server device 12.

[Operations and Effects of Second Embodiment]

According to the diet plan management method of the second embodiment, the following operations and effects can be obtained.

[1]

In a case where the measurement of the third physical information is delayed by a prescribed number of times or more, the server device 12 transmits the drop concern warning to the target person terminal device 24. The drop concern warning screen 300 indicating the drop concern warning is displayed on the display device of the target person terminal device 24. Accordingly, the target person can grasp that the measurement of the third physical information is delayed by the prescribed number of times or more.

[2]

The selection information for selecting whether or not to change the diet plan can be transmitted from the target person terminal device 24 to the server device 12 by using the drop concern warning screen 300.

Although it has been described in the second embodiment that the drop concern warning is transmitted based on the delay of the third physical information measurement, it is also possible to transmit the drop concern warning based on the delay of the blood inspection.

### Third Embodiment

[Description of Problems]

The diet based on a genetic inspection has been known. In a case where the diet plan is created, in the diet based on the genetic inspection, weight gain of the target person is grasped based on the inspection result of the genetic inspection. Accordingly, it is possible to create the diet plan corresponding to the weight gain of the target person.

It is also known that a change in a distribution of intestinal flora influences adult diseases such as obesity, lifestyle-related disease, and cancer. It is also known that the distribution of the intestinal flora is changed especially in older ages. The distribution of the intestinal flora can be grasped by using culture. In recent years, the distribution of the intestinal flora can be grasped by genetic analysis using a polymerase chain reaction. The polymerase chain reaction may be referred to as PCR.

However, all these inspections are effective information in a case where the diet plan is created, but are difficult to be applied to the management of the diet plan such as grasping the progress of the diet plan. Thus, in a diet plan management method according to a third embodiment, the diet plan is created by using, as an option inspection, at least one of the genetic inspection or the intestinal flora inspection in combination with the blood inspection, and the progress of the diet plan is managed by using the blood inspection.

[Procedure of Diet Plan Management Method According to Third Embodiment]

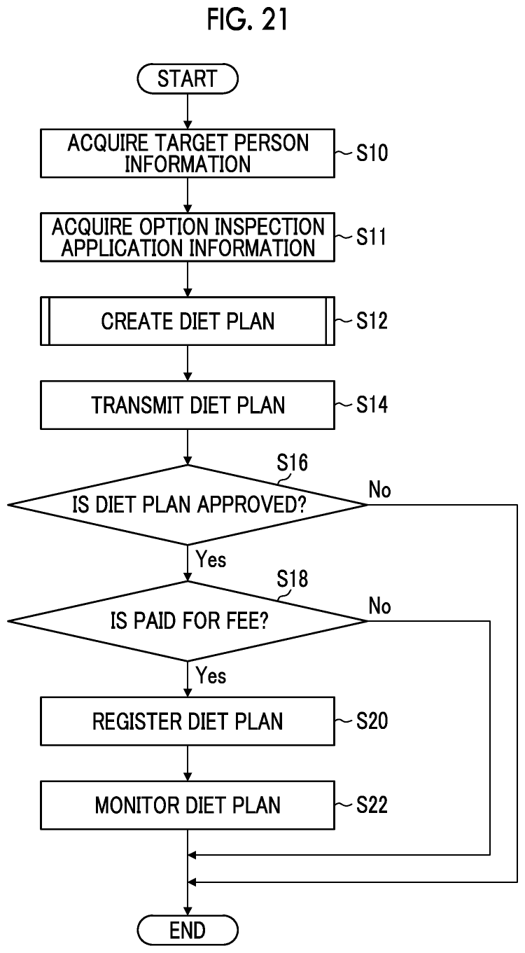

FIG. 21 is a flowchart showing a procedure flow of the diet plan management method according to the third embodiment. In the flowchart of the diet plan management method shown in FIG. 21, option inspection application information acquisition step S11 is added to the flowchart of the diet plan management method shown in FIG. 6.

In option inspection application information acquisition step S11, the server device 12 acquires option inspection application information transmitted from the target person terminal device 24. At least one of the genetic inspection or the intestinal flora inspection is applied as the option inspection.

In a case where the option inspection application information is acquired, the server device 12 acquires a result of the option inspection corresponding to option inspection application information before diet plan creation step S12 is executed. The server device 12 creates the diet plan by using the result of the option inspection together.

[Configuration Example of Option Inspection Application Screen]

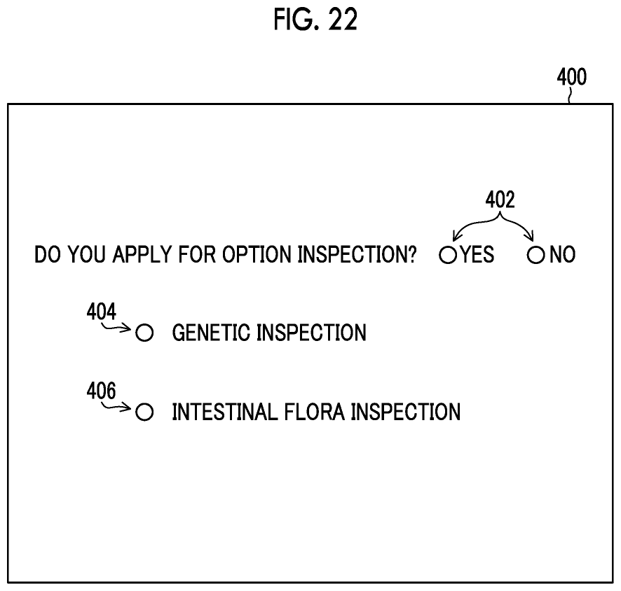

FIG. 22 is an explanatory diagram showing an example of an option inspection application screen. An option inspection application screen 400 shown in FIG. 22 is displayed on the display device connected to the target person terminal device 24. The option inspection application screen 400 includes an application selection button 402, a genetic inspection selection button 404, and an intestinal flora inspection selection button 406.

The target person can select whether or not to apply for the option inspection by using the option inspection application screen 400. The target person can select at least one of the genetic inspection or the intestinal flora inspection by using the option inspection application screen 400.

[Genetic Inspection]

An example of the genetic inspection is an example in which blood is collected as a sample and the genetic inspection is performed by using white blood cells in the blood is illustrated. Genetic analysis related to the obesity in the genetic inspection is performed, and obesity information is created based on the result of the genetic analysis.

Other examples of the genetic inspection include a genetic inspection in which saliva is collected as a sample and a component of the saliva is used and a genetic inspection in which cells such as mucous membrane are collected as a sample and components of the cells are used.

[Intestinal Flora Inspection]

An example of the intestinal flora inspection is an intestinal flora inspection in which stool is collected as a sample and the number, ratio, and distribution of Bacteroides, Bifidobacteria, Escherichia coli, Lactobacillus, and C. perfringens are analyzed.

[Operations and Effects of Third Embodiment]

According to the diet plan management method of the third embodiment, the following operations and effects can be obtained.

[1]

The diet plan is created by using at least one of the inspection result of the genetic inspection or the inspection result of the intestinal flora inspection together. Accordingly, it is possible to create the diet plan by using the information of the target person grasped from at least one of the inspection result of the genetic inspection or the inspection result of the intestinal flora inspection together.

[2]

The option inspection may be implemented only once in a case where the diet plan is created.

[Application Example to Network System]

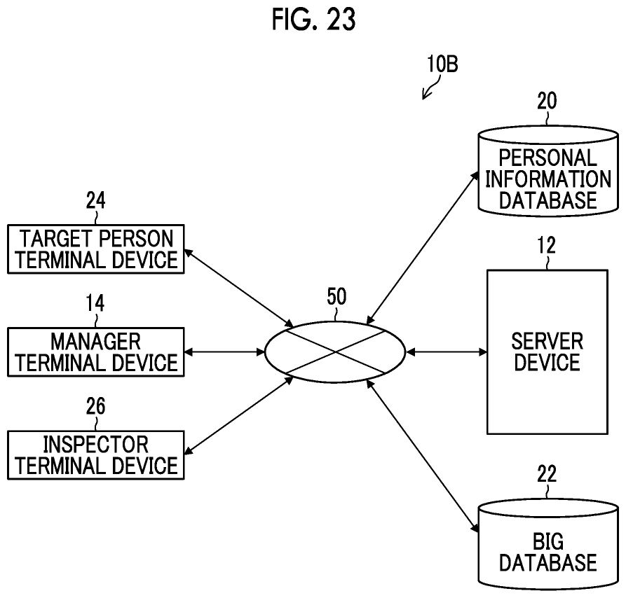

FIG. 23 is a block diagram showing a configuration example of a diet plan management system to which the network system is applied. A diet plan management system 10B shown in FIG. 23 is configured such that the server device 12 can be connected to the network 50. In the diet plan management system 10B, the personal information database 20 and the big database 22 are connected to the server device 12 via the network 50 so as to perform data communication.

In the diet plan management system 10B, the manager terminal device 14, the target person terminal device 24, and the inspector terminal device 26 are connected to the server device 12 via the network 50 so as to perform data communication.

A public line network or a dedicated line network may be applied to the network 50. A high-speed communication cable such as an optical fiber can be applied to the connection between the network 50 and each device.

The server device 12 and the manager terminal device 14 may be directly connected without the network 50. The server device 12 and the personal information database 20 may be directly connected without using the network 50.

[Specific Example of Sample Inspection Kit]

Specific examples of the blood inspection kit include, for example, a biological sample separation device described in the specification of JP3597827B and a sample analysis tool described in the specification of JP3643011B. The blood inspection kit may further include treatment tools such as a lancet, a bandage, and a disinfectant cloth, and an instruction manual used in a case where the blood is collected. It is possible to acquire the inspection result by delivering the biological sample separation device and the sample analysis tool between a patient who collects blood and an inspection facility by home delivery or by mail.

[Operations and Effects of Blood Collection Using Blood Inspection Kit]

According to the blood collection using the blood inspection kit described above, the following operations and effects can be obtained.

[1]

The blood can be collected at homes other than medical institutions.

[2]

Since the blood inspection kit is delivered on a prescribed blood collection date, forgetting to collect blood and mistakes in the blood collection date can be suppressed.

[3]

The amount of collected blood is as small as 0.1 ml or less.

[Application Example to Program Causing Computer to Function as Diet Plan Management Device]

The above-described diet plan management method can be configured as a program causing a computer to realize a function corresponding to each unit in the diet plan management device and a function corresponding to each step in the diet plan management method.

For example, a diet plan management program can cause a computer to realize a physical information acquisition function, a blood inspection item value acquisition function, a diet plan creation function, and a diet plan monitoring function.

It is possible to store the program causing the computer to realize the above-described diet plan management function in a computer-readable information storage medium which is a tangible non-transitory information storage medium, and it is possible to provide the program through the information storage medium.

Instead of an aspect of storing the program in the non-transitory information storage medium and providing the program, an aspect of providing a program signal via a network can also be realized.

[Combinations of Embodiments and Modification Examples]

The components described in the above-described embodiment and the components described in the modification examples can be appropriately used in combination, and some of the components can be replaced.

In the above-described embodiment of the present invention, components can be appropriately changed, added, or deleted without departing from the spirit of the present invention. The present invention is not limited to the above-described embodiments, and can be variously modified by those having ordinary knowledge in the art within the technical spirit of the present invention.

### EXPLANATION OF REFERENCES

10: diet plan management system
10A: diet plan management system
10B: diet plan management system
12: server device
12A: server device
14: manager terminal device
20: personal information database
20A: personal information database
22: big database
24: target person terminal device
26: inspector terminal device
30: information acquisition unit
30A: physical information acquisition unit
30B: blood inspection item value acquisition unit
32: information output unit
32A: plan information output unit
32B: delivery information output unit
32C: monitoring information output unit
34: planning unit
36: analysis unit
38: monitoring unit
40: controller
42: memory
44: storage device
46: network controller
48: power supply device
50: network
52: bus
100: application screen
102: name input field
104: gender input field
106: date of birth input field
108: age input field
110: height input field
112: weight input field
114: BMI value input field
116: body fat percentage input field
120: blood inspection result presence and absence selection button
122: first desired course selection button
124: second desired course selection button
126: third desired course selection button
128: kit delivery destination selection button
130: kit delivery destination input field
150: diet plan proposal screen
152: target person information display field
154: blood inspection result presence and absence display field
156: desired course display field
158: blood inspection kit delivery destination display field
160: first physical information measurement condition display field
162: blood inspection condition display field
164: approval button
166: change button
180: diet acceptance screen
182: acceptance completion information
184: OK button
200: weight information input screen
202: target person name display field
204: weight input field
206: OK button
220: progress report screen
220A: progress report screen
222: target person name display field
224: blood inspection item value display field
226: achievement level display field
228: comment display field
230: course change selection button
300: drop concern warning screen
302: target person name display field
304: drop concern warning display field
306: selection button
400: option inspection application screen
402: application selection button
404: genetic inspection selection button
406: intestinal flora inspection selection button
S10 to S548: each step of diet plan management method

What is claimed is:

1. A diet plan management device comprising:
a physical information acquisition unit that acquires physical information of a target person including at least one of a weight, a BMI value, or a body fat percentage of the target person;
a blood inspection item value acquisition unit that acquires a blood inspection item value of the target person;
a diet plan creation unit that changes, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creates a diet plan for implementing one or more blood inspections; and
a diet plan monitoring unit that monitors the diet plan based on a third blood inspection item value which is acquired by using the blood inspection item value acquisition unit and is acquired as a result of the blood inspection.

2. The diet plan management device according to claim 1,
wherein the diet plan creation unit creates the diet plan for implementing one or more measurements of the physical information, and
the diet plan monitoring unit monitors the diet plan based on third physical information acquired as a result of the measurement of the physical information.

3. The diet plan management device according to claim 2, further comprising:
a physical information storage unit that stores the third physical information acquired from the measurement of the physical information.

4. The diet plan management device according to claim 1,
wherein the physical information acquisition unit acquires the second physical information, and
the diet plan creation unit creates the diet plan by using the acquired second physical information.

5. The diet plan management device according to claim 1,
wherein the diet plan creation unit creates the diet plan by using prescribed second physical information.

6. The diet plan management device according to claim 2,
wherein the diet plan creation unit decides at least one of an implementation period in which the diet plan is implemented, a measurement frequency of the third physical information, or an implementation frequency of the blood inspection.

7. The diet plan management device according to claim 2, further comprising:
an analysis unit that creates progress status information indicating a progress status of the diet plan by analyzing at least one of the third physical information or the third blood inspection item value.

8. The diet plan management device according to claim 7, further comprising:
a progress status information output unit that outputs the progress status information.

9. The diet plan management device according to claim 7,
wherein the analysis unit generates change proposal information indicating a change proposal of the diet plan based on the progress status information.

10. The diet plan management device according to claim 9, further comprising:
a change proposal information output unit that outputs the change proposal information.

11. The diet plan management device according to claim 6, further comprising:
a delivery information output unit that transmits, to a delivery source of a blood inspection kit, blood inspection kit delivery information for delivering the blood inspection kit used for the blood inspection to the target person based on the implementation frequency of the blood inspection.

12. The diet plan management device according to claim 6,
wherein, in a case where the input of the third physical information based on an input frequency of the third physical information is not implemented, the diet plan monitoring unit creates encouragement information for encouraging the input of the third physical information.

13. The diet plan management device according to claim 12, further comprising:
an encouragement information output unit that outputs the encouragement information.

14. The diet plan management device according to claim 1,
wherein the diet plan creation unit creates the diet plan by using a result of an intestinal flora inspection together.

15. The diet plan management device according to claim 1,
wherein the diet plan creation unit creates the diet plan by using a result of a genetic inspection together.

16. The diet plan management device according to claim 1,
wherein the blood inspection item value acquisition unit acquires, as the first blood inspection item value, the second blood inspection item value, and the third blood inspection item value, at least one item value of blood sugar, HbA1c, HDL cholesterol, LDL cholesterol, neutral fat, AST, ALT, γ-GPT, ALB, ALP, or e-GFR.

17. A diet plan management system comprising a server device configured to be connected to a network,
wherein the server device includes
a physical information acquisition unit that acquires physical information of a target person including at least one of a weight, a BMI value, or a body fat percentage of the target person;
a blood inspection item value acquisition unit that acquires a blood inspection item value of the target person;
a diet plan creation unit that changes, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creates a diet plan for implementing one or more blood inspections; and
a diet plan monitoring unit that monitors the diet plan based on a third blood inspection item value which is acquired by using the blood inspection item value acquisition unit and is acquired as a result of the blood inspection.

18. The diet plan management system according to claim 17, further comprising:
a physical information storage device that is connected to communicate with the server device via the network,
wherein the physical information storage device stores the physical information obtained from a measurement of the physical information.

19. The diet plan management system according to claim 17, further comprising:
a blood inspection item value storage device that is connected to communicate with the server device via the network,
wherein the blood inspection item value storage device stores the blood inspection item value associated with the physical information.

20. The diet plan management system according to claim 19,
wherein the blood inspection item value storage device stores a relationship between at least one of personal information, hobby and taste information regarding hobby and taste, living environment information regarding a living environment, or family information regarding a family and the blood inspection item value.

21. The diet plan management system according to claim 17, further comprising:
a terminal device that is connected to communicate with the server device via the network,
wherein the terminal device includes a display unit that displays information regarding the diet plan of the target person, and
the display unit displays, as the information regarding the diet plan, at least one of an application screen for applying for the diet plan, a proposal screen indicating a proposal content of the diet plan, or a progress screen indicating a progress of the diet plan.

22. A diet plan management method comprising:
a physical information acquisition step of acquiring physical information of a target person including at least one of a weight, a BMI value, or a body fat percentage of the target person;
a blood inspection item value acquisition step of acquiring a blood inspection item value of the target person;
a diet plan creation step of changing, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creating a diet plan for implementing one or more blood inspections; and
a diet plan monitoring step of monitoring the diet plan based on a third blood inspection item value which is acquired in the blood inspection item value acquisition step and is acquired as a result of the blood inspection.

23. A non-transitory computer readable recording medium causing a computer to realize a function in a case where a command stored in the recording medium is read by the computer, the function comprising:
a physical information acquisition function of acquiring physical information of a target person including at least one of a weight, a BMI value, or a body fat percentage of the target person;
a blood inspection item value acquisition function of acquiring a blood inspection item value of the target person;
a diet plan creation function of changing, based on a relationship between a first blood inspection item value corresponding to first physical information which is the physical information before the target person implements a diet and a second blood inspection item value corresponding to second physical information which is target physical information of the target person, the physical information of the target person to the second physical information from the first physical information and creating a diet plan for implementing one or more blood inspections; and
a diet plan monitoring function of monitoring the diet plan based on a third blood inspection item value which is acquired by using the blood inspection item value acquisition function and is acquired as a result of the blood inspection.
